(19) United States
(12) Patent Application Publication
(10) Pub. No.: US 2020/0069778 A1
(43) Pub. Date: Mar. 5, 2020

(54) PLANT-EXPRESSED COCAINE HYDROLASE VARIANTS OF BUTYRYLCHOLINESTERASE AND METHODS OF REDUCING COCAINE-PRIMED REINSTATEMENT

(71) Applicant: ARIZONA BOARD OF REGENTS ON BEHALF OF ARIZONA STATE UNIVERSITY, Scottsdale, AZ (US)

(72) Inventors: Tsafrir Mor, Tempe, AZ (US); Katherine Larrimore, Singapore (SG); Ismail (John) Kazan, Tempe, AZ (US); Sefika Ozkan, Tempe, AZ (US); Janet Neisewander, Scottsdale, AZ (US)

(21) Appl. No.: 16/560,993

(22) Filed: Sep. 4, 2019

Related U.S. Application Data
(60) Provisional application No. 62/726,924, filed on Sep. 4, 2018.

(51) Int. Cl. A61K 38/46; A61P 25/36

(52) U.S. Cl. CPC A61K 38/465; A61P 25/36; C12Y 301/01008

(57) ABSTRACT

The disclosure relates to the use of cocaine hydrolase variants of butyrylcholinesterase (BChE) in treating nerve gas exposure or as protection against anticholinesterases such as nerve agents. The disclosure includes particular methods of use, including a method of reducing cocaine-primed reinstatement of drug-seeking behavior in a mammalian subject having had prior exposure to cocaine.

### CROSS REFERENCE TO RELATED APPLICATIONS

This application claims the benefit of and priority to U.S. provisional patent application 62/726,924, filed Sep. 4, 2018, the entirety of the disclosure of which is hereby incorporated by this reference.

### TECHNICAL FIELD

The disclosure relates to plant-expressed cocaine hydrolase variants of butyrylcholinesterase and methods of reducing cocaine-primed reinstatement.

### BACKGROUND

Cocaine abuse is a global problem with major medical and societal consequences. Cocaine-use disorders include short- and long-term pathologies (for example, overdose, intractable addiction, and post-abstinence relapse) that affect millions of people worldwide with no available treatment to effectively reduce morbidity and mortality associated with such disorders. Patients suffering from acute toxicity (overdose) are only symptomatically treated and there is no effective FDA-approved treatment to decrease the likelihood of relapse in rehabilitated addicts. One therapeutic approach for counteracting the toxic and addictive psychoactive effects of cocaine is to accelerate the drug's metabolism, for example, using enzymes that can break down active cocaine molecules into inactive metabolites.

Butyrylcholinesterase (BChE) is a human serum enzyme that is capable of binding and/or hydrolyzing a diverse array of compounds including many natural and man-made toxicants of the central and peripheral nervous system, unlike the highly selective homologous enzyme acetylcholinesterase (AChE). BChE is capable of counteracting the toxicity of various anticholinesterases by binding to them before they reach their targets in the nervous system. BChE can detoxifying organophosphorous (OP) nerve agents like paraoxon, as well as acetylcholine receptor antagonists, and psychoactive plant alkaloids such as cocaine. Exogenously supplied BChE can augment the bioscavenging capacity of the endogenous enzyme and provide broad protection by sequestering the anticholinesterase agents.

In humans, BChE is responsible for the inactivation of cocaine, resulting in the transient nature of cocaine-induced euphoria. However, the limited hydrolytic capacity of this promiscuous enzyme toward (−)-cocaine, the psychoactive enantiomer of the drug, does not allow the enzyme to remove it rapidly enough from the human body in situations of overdose. Mutants of BChE have been rationally designed (for example, using molecular dynamics and modeling based on the atomically resolved structure of the enzyme (PDB 1P0P)) to create highly efficient recombinant cocaine hydrolases compared to the wild-type (WT) BChE (UniProt accession number P06276). The variants were designed to be an enzyme-based therapy to treat drug overdose and addiction.

One such cocaine hydrolase BChE variant (A199S/S287G/A328W/Y332G), when derived from mammalian expression systems, has been shown to accelerate cocaine metabolism in vivo and to fully protect mice and rats from respective lethal doses of cocaine. This enzyme has also been shown to be safe and effective in humans to accelerate cocaine metabolism in Phase I and Phase II clinical trials. In an effort to further improve the efficiency of cocaine hydrolysis, a pentavalent mutant (A199S/F227A/S287G/A328W/Y332G) was designed, and it was shown to be an even more effective cocaine hydrolase both in vitro and in vivo.

The catalytic activity of WT hBChE against cocaine is measurable, albeit slow, and provides one of the major detoxification pathways for the drug, generating non-psychoactive metabolites. When designing BChE-based cocaine hydrolase mutants, care was taken to ensure that their ability to hydrolyze the crucially important substrate, acetylcholine (ACh), was not significantly enhanced. While these highly efficient cocaine-metabolizing variants of BChE were designed with the goal of increasing catalytic efficiency of cocaine hydrolysis toward an anti-cocaine treatment, whether the newly introduced mutations can affect the role of BChE in interfering with the drug reinstatement neurological pathway remains unclear.

### SUMMARY

The disclosure relates to the use of a cocaine hydrolase variant of human serum butyrylcholinesterase (BChE, UniProt accession number P06276) to reduce cocaine-primed reinstatement of drug-seeking behavior in mammals having had prior exposure to cocaine or to treat acute cocaine toxicity. In one useful embodiment, the cocaine hydrolase variant of BChE is plant-expressed and a pentavalent mutant with a substitution mutation at residue 199, residue 227, residue 287, residue 328, and residue 332. In certain beneficial embodiments, the cocaine hydrolase variant of BChE have the following substitution mutations: A199S, F227A, S287G, A328W, and Y332G. In some aspects, the plant-expressed BChE is a tetramer and/or comprises a C-terminal 6x histidine tag.

In certain implementations, the cocaine hydrolase variant of BChE is administered parenterally, for example, intravenously, sublingually, subcutaneously, or transdermally. The cocaine hydrolase variant of BChE can also be administered via a pulmonary route. The amount of the cocaine hydrolase variant of BChE administered is typically at least 3 mg/kg body weight of the mammalian subject. In certain particular aspects, the amount of the cocaine hydrolase variant of BChE administered is 3 to 10 mg/kg body weight, e.g., about 3 mg/kg body weight of the mammalian subject.

The mammalian subject being treated has often had multiple self-induced exposures to cocaine. For example, the mammalian subject is an addict. As used herein, the term multiple exposures means more than once, e.g., at least 5 times, at least 10 times, etc. In other implementations, the mammalian subject may be administered multiple doses of cocaine by another party. The cocaine-seeking behavior of the mammalian subject, in certain embodiment, has been extinguished for at least one day, at least one week, or at least two weeks.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1H depict, in accordance to certain embodiments, schematics of plant production of a cocaine hydrolase variant of BChE and biochemical characterization of the plant-derived cocaine hydrolase variant of BChE (pBChE). FIGS. 1A and 1E depict an exemplary plant-based strategy for the production of BChE. Plant-expression optimized synthetic genes encoding human BChE and variants thereof were cloned into the TMV-based MagnICON vector system, which recombines in vivo to yield a cell-to-cell-spreading replicon. WT Nicotiana benthamiana plants were infiltrated with agrobacteria harboring the MagnICON vectors, and on peak accumulation day of the transiently expressed recombinant enzymes, leaf material was harvested, homogenized and the enzymes were purified. Following dialysis into a suitable buffer, the purified PCocSH product can be stored for several years at 4° C. and used for testing. Transient expression replicon: RpRd, RNA-dependent RNA polymerase; MP, movement protein gene; a, barley alpha-amylase signal peptide. Wavy lines represent the translation products of the replicon genes. FIG. 1B depicts an exemplary purification result of pBChEᵥ₄. Leaf extract from pBChEᵥ₄-expressing plants was clarified by 70% (NH₄)₂SO₄ precipitation then subject to ConA purification and eluted with stepwise increasing concentrations of methyl-α-D-mannopyranoside ([E1]-[E5]). Samples from these purification steps, protein size markers (M) and an un-infiltrated WT N. benthamiana extract control (C) were subject to SDS-PAGE followed by silver-staining (top) or BChE-specific immunoblotting (bottom). Lanes in respective gels were loaded based on equal enzymatic activity. FIG. 1C depicts an exemplary oligomerization of pBChEᵥ₄. Purified preparation of pBChEᵥ₄ was analyzed by SEC-HPLC; fractions were monitored for total protein content (top) and pooled fractions (0.5 mL every 1 min) for enzymatic activity (bottom). Inset: fractionation pattern of WT pBChE. Molecular mass standards are indicated with arrows. FIG. 1D depicts exemplary enzymatic hydrolysis of (−)-cocaine by WT pBChE and pBChEᵥ₄. Purified samples of WT pBChE (green, 1.21×10⁻¹ upper and lower panel) and pBChEᵥ₄ (pink, 6.06×10⁻⁴ upper panel). Curves represent nonlinear regression fitted to the Michaelis-Menten model (Equation 1). Fitting the data to the Radić model (substrate inhibition, Equation 2) does not result in a significantly better fit (based on the extra sum-of-squares F test; p>0.12 and p>0.78 for the mutant and WT enzymes, respectively). FIGS. 1E and 1F respectively depict size exclusion-HPLC analysis and enzymatic activity of fractions collected during SE-HPLC analysis. Monomer, dimer, and tetramer fractions are indicated with 1°, 2°, and 4° respectively. Blue dextran (2000 kDa), beta amylase (200 kDa), albumin (66 kDa) and carbonic anhydrase (29 kDa) were included as size references and respective peak elution times are indicated by arrows. Samples for enzymatic activity were collected from the HPLC (0.5 mL) every minute. FIGS. 1G and 1H respectively depict silver stain and western blot of pooled, purified preparation of PCocSH used in animal studies alongside uninfiltrated wildtype (WT) Nicotiana benthamiana control plant extract.

FIGS. 2A and 2B depict, in accordance to certain embodiments, BTC hydrolysis by WT hBChE, WT pBChE, and pBChEᵥ₂₋₅. FIG. 2A plots reaction rates against substrate concentration (mean±SEM). Plots in FIG. 2B zoom in on the low range of substrate concentrations. The 100% values and the goodness of fit values are as follows:

WT hBChE, 100%=1.57±0.04 nmol/min, Equation (2), R2=0.98;

WT pBChE, 100%=1.21±0.07 nmol/min, Equation (2), R2=0.99;

pBChEᵥ₂, 100%=0.79±0.10 nmol/min, Equation (3), R2=0.83;

pBChEᵥ₃, 100%=0.96±0.01 nmol/min, Equation (3), R2=0.95;

pBChEᵥ₄, 100%=8.9±0.6 nmol/min, Equation (3), R2=0.95;

pBChEᵥ₅, 100%=8.1±0.3 nmol/min, Equation (3), R2=0.95

FIGS. 3A and 3B depict, in accordance to certain embodiments, ATC hydrolysis by WT hBChE, WT pBChE, and pBChEV₂₋₅. FIG. 3A plots reaction rates are plotted against substrate concentration (mean±SEM). Plots in FIG. 3B zoom in on the low range of substrate concentrations. The 100% values and the goodness of fit values are as follows:

WT hBChE, 100%=0.97±0.04 nmol/min, Equation (2), R2=0.99;

WT pBChE, 100%=3.0±0.1 nmol/min, Equation (2), R2=1.00;

pBChEᵥ₂, 100%=4.7±0.1 nmol/min, Equation (2), R2=0.99;

pBChEᵥ₃, 100%=1.39±0.00 nmol/min, Equation (3), R2=0.98;

pBChEᵥ₄, 100%=2.6±0.1 nmol/min, Equation (1), R2=0.94;

pBChEᵥ₅, 100%=12.0±0.7 nmol/min, Equation (3), R2=0.99

FIGS. 4A and 4B depict, in accordance to certain embodiments, the % DCI profile of WT hBChE. The % DCI profiles for hBChE are color-coded in a cartoon diagram from a spectrum of red-white-blue (red -highest, blue -lowest coupling to perturbation locations). FIG. 5A shows that, upon perturbation of catalytic residues (S198, E325, and H438 shown as grey sticks), the five mutation positions (A199, F227, S287, A328, and Y332 shown as red sticks) shows high coupling (high % DCI values). FIG. 4B shows that upon perturbation of five mutation positions (A199, F227, S287, A328, and Y332 shown as grey sticks), the catalytic residues (S198, G325, and H438 shown as red sticks) shows high coupling (high % DCI values).

FIGS. 5A-5E depicts the % DFI profile of WT hBChE and pentavalent mutant. FIG. 5A shows the % DFI profiles of WT BChE (blue) and BChEV4 (x-axis—residue numbers, y-axis-% DFI values at each position). FIG. 5B contains the color-coded structure diagrams depicting the % DFI values at each position. The circled regions are part of the monomer-monomer contact region (V377, D378, T457, K458, A459, 1462, Y500, R509, M511, T512, K513, R514, L515). FIG. 5C shows the % DFI profiles of monomeric (blue) and dimeric (green) WT BChE. FIG. 5D shows the % DFI profiles of monomeric (red) and dimeric (purple) BChEᵥ₄. FIG. 5E contains the color-coded structure diagrams depicting the values of % DFI differences between the dimeric forms of WT BChE and BChEV4 at each position. The red-white-blue code reveals loci with increased flexibility (shades of red), decreased flexibility (shades of blue) or no change (white) in the mutant dimer vs. the WT dimer.

FIGS. 6A and 6B contain the schematic diagrams describing the kinetics of cholinesterase-catalyzed hydrolysis of substrates. FIG. 6A shows Scheme 1, which describes the reaction of cholinesterase (E)-catalyzed hydrolysis of substrates (S). Kₛₛ is the dissociation constant of the peripheral site. The hydrolysis capacity (bK_{cat}) reflects the allosteric effect of substrate binding at the peripheral binding site. FIG. 6B shows Scheme 2, which describes the reaction in terms of uncompetitive substrate inhibition/activation and cooperative substrate binding with characteristic Hill coefficients n and x that describe cooperativity or anticooperativity.

FIG. 7 depicts the Elastic Network Model (ENM) of WT human BChE and BChEV4. The spheres indicate the locations of alpha carbons of each amino acid and the sticks are representing the harmonic oscillators (i.e. springs) between them. The thickness of the sticks represents the magnitude of the spring constant. For WT hBChE the mutation positions are shown as blue spheres (A199, F227, S287, A328, and Y332) and for the pentavalent mutant BChEᵥ₄ the mutation positions are shown as red spheres (S199, A227, G287, W328, and G332). The spring constant for each connection is assumed to be same for the WT (i.e. the thickness of the blue sticks is same as grey sticks). The mutation at a given position are considered to destabilize the interactions of the mutational site. This is incorporated in the model as a decrease in spring constant (low thickness values are shown as red sticks indicating a loss in interaction strength with mutated positions).

In accordance with certain embodiments, FIGS. 8A-8C show that PCocSH offers protection from cocaine-induced toxicity. FIG. 8A depicts the timeline of an exemplary protection experiment. PCocSH (3 or 10 mg/kg) or vehicle control was delivered (i.v.) 5 minutes prior to the administration of varying doses of cocaine (i.p). Acute symptoms in control animals arose within the first 5 minutes following the cocaine challenge. Experiment was concluded 60 minutes post cocaine administration or upon moribundity (see Methods). FIG. 8B depicts the dose response curve of cocaine-induced convulsions and tremors, and FIG. 8C depicts the dose response curve of cocaine-induced lethality. Each data point represents the % of mice (N=6, unless otherwise noted) for each dosing condition.

In accordance with certain embodiments, FIGS. 9A-9D show administration of PCocSH can rescue a subject exposed to an overdose of cocaine from cocaine-induced toxicity. FIG. 9A depicts the timeline of an exemplary rescue experiment. Experiment was concluded 60 minutes post cocaine administration or upon moribundity. FIG. 9B shows the severity of the symptom displayed by each mouse at the end of the exemplary 60-minute experiment. Symptoms were scored 0 for asymptomatic, 1 for decreased motor activity, 2 for tremors or fasciculation, 3 for convulsions, and 4 for death. Individual mice (represented by symbols) were i.p. injected with cocaine in saline (100 mg/kg). Within approximately 1 minute of onset of cocaine-induced seizures, PCocSH at 3 mg/kg (blue) or 10 mg/kg (red) or vehicle control (black) was delivered i.v., and animals were observed for 60 minutes. The asterisks represent a significant difference from the condition of mice treated with PBS (*p<0.05, **p<0.01). Methods section provides details. FIG. 9C depicts survival curves of mice treated with vehicle control (N=5), PCocSH 3 mg/kg (N=6) or 10 mg/kg (N=5). Comparison by the Log-rank (Mantel-Cox) test between low dose and control (p=0.0016) and high dose and control (p=0.0027) were significant. FIG. 9D depicts the time to recovery from cocaine-induced seizures following PCocSH treatment. Time (in minutes) from the onset of cocaine-induced convulsions until upright posture and normal motor activity resumed is shown. Comparison by the Log-rank (Mantel-Cox) test between low dose and control (p=0.001) and high dose and control (p=0.0016) were significant.

In accordance with certain embodiments, FIGS. 10A and 10B show that administration of PCocSH can prevent cocaine-seeking reinstatement behavior in mice. FIG. 10A depicts a timeline of an exemplary conditioned place preference (CPP) experiment (top) and diagram of chambers used in CPP studies (bottom). During times of unrestricted access, mice had free access to both compartments of the chamber without a middle partition. During conditioning, saline or cocaine (10 mg/kg) injections (i.p.) were given immediately prior to placing animals in the conditioning chambers. Mice received either saline and were placed in the non-drug-paired compartment or received cocaine (10 mg/kg, i.p.) and were placed in the drug-paired compartment. During extinction of cocaine-induced CPP, mice received saline only and on alternating days were placed in either non-drug-paired compartment or drug-paired compartment. FIG. 10B depicts the time spent in initially least preferred side over the course of the exemplary CPP study. Following extinction of cocaine-seeking behavior, mice were either treated with vehicle (top panel) or PCocSH (3 mg/kg) (bottom panel) prior to receiving a priming dose of cocaine (10 mg/kg) or saline. *Different from baseline, paired samples t-test with Bonferroni correction, P<0.01, #Different from CPP test, paired samples t-test with Bonferroni correction, P<0.01, +Different from extinction, planned comparison, P<0.05.

### DETAILED DESCRIPTION

Detailed aspects and applications of the disclosure are described below in the following drawings and detailed description of the technology. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the disclosure. It will be understood, however, by those skilled in the relevant arts, that embodiments of the technology disclosed herein may be practiced without these specific details. It should be noted that there are many different and alternative configurations, devices and technologies to which the disclosed technologies may be applied. The full scope of the technology disclosed herein is not limited to the examples that are described below.

The singular forms “a,” “an,” and “the” include plural referents unless the context clearly dictates otherwise. Thus, for example, reference to “a step” includes reference to one or more of such steps.

As used herein, the term “about” refers to a deviation up to but not more than 10% of the given value, for example a deviation of 10%, 7.5%, 5%, 4%, 3%, 2%, 1%, 0.5%, or 0.1% of the given value.

As used herein, the term “subject,” “mammalian subject,” or “addict,” refers to any mammalian subject or patient to which the methods of the invention can be applied. “Mammals” refers to human patients and non-human primates, as well as experimental animals such as rabbits, rats and mice, and other animals. The term “addict” also refers to a mammalian subject who has had multiple exposures to cocaine, whether self-induced or not self-induced. In particular embodiment, the term “addict” refers to a mammalian subject that is administered cocaine on a regular basis, whether self-administered or by another.

As used herein, the amino acid sequence of wild-type human serum butyrylcholinesterase (BChE) is set forth in UniProt accession number P06276.

As used herein, the term “cocaine hydrolase variants of butyrylcholinesterase” are BChEs altered to have cocaine hydrolase activity. Examples of such mutated BChE include BChE with the following combination of substitution mutations: F227A, S287G, A328W, and Y332A (also referred to herein as pBChEᵥ₂ when produced in plants); A199S, S287G, A328W, and Y332G (also referred to herein as pBChEᵥ₃ when produced in plants); A199S, F227A, S287G, A328W, and Y332G (also referred to herein as pBChEᵥ₄ or PCocSH (Plant-derived form of the Cocaine Super Hydrolase) when produced in plants); F227A, S287G, A328W, and Y332G (also referred to herein as pBChEᵥ₅ when produced in plants).

The term “plant-derived cocaine hydrolase variants of BChE” (pBChE, as opposed to the wild-type human serum BChE abbreviated as hBChE) as used herein, refers to any cocaine hydrolase variant of BChE that are produced from plants expressing a gene encoding a cocaine hydrolase variant of BChE. For example, the cocaine hydrolase variants of BChE are produced by a plant with its genome modified to express a synthetic gene encoding the cocaine hydrolase variant of BChE. In certain embodiments, the gene may be codon optimized for plant expression.

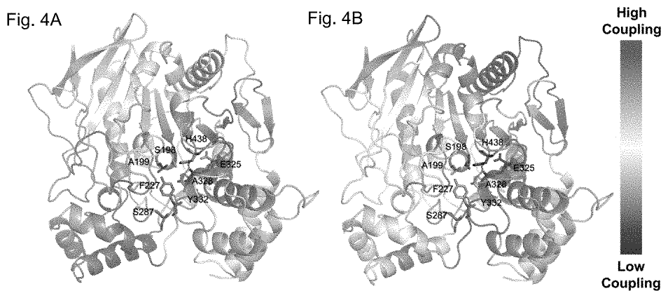

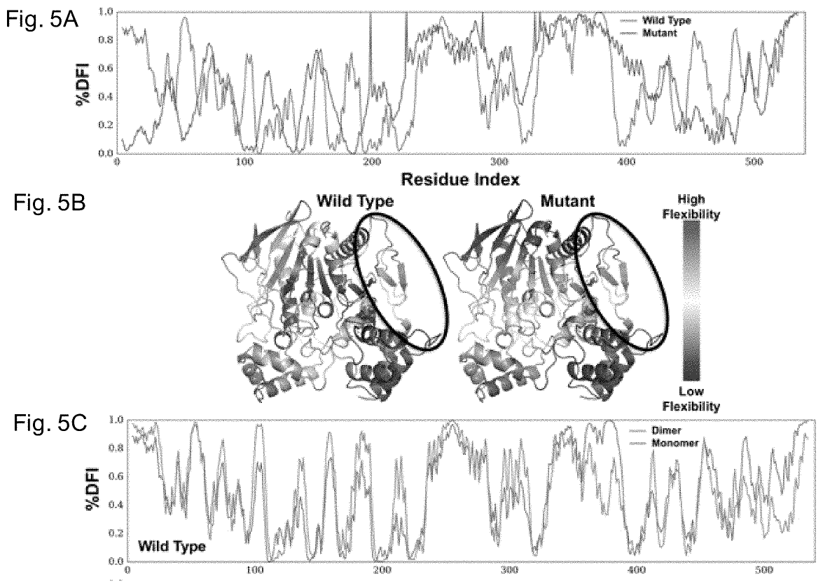

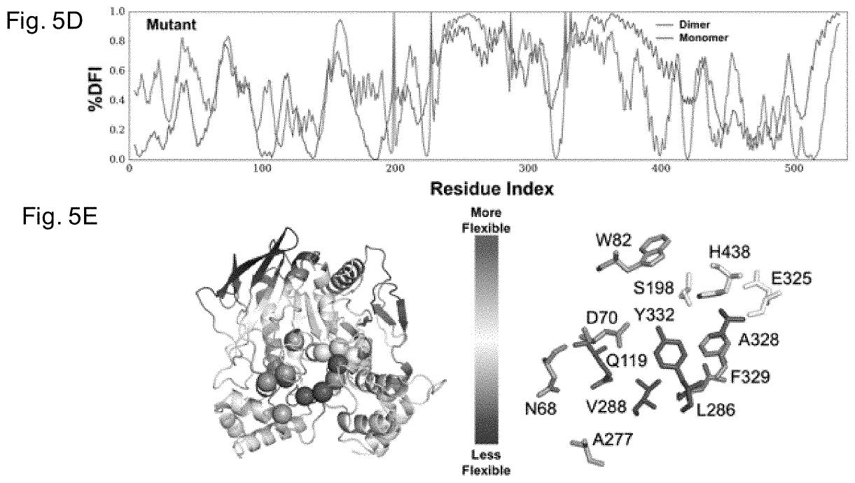

The disclosure relates to the discovery that the mutations at residue 119, residue 227, residue 287, residue 328, and residue 332 of BChE allosterically affect the catalytic triad not only within a single subunit but also propagate to neighboring subunits of the BChE oligomer (see, for example, FIGS. 4A-5B). The disclosure also relates to the discovery that a plant-derived cocaine hydrolase variant of BChE, when administered to a mammalian subject having had prior exposure to cocaine, rescues the subject from toxic effects of cocaine exposure, such as in the case of an overdose, and also reduces cocaine-induced reinstatement of drug-seeking behavior. The likelihood that a subject undergoing cocaine addiction treatment will relapse can be reduced by the administration of a pBChE. In other words, the plant-derived cocaine hydrolase variants described herein are useful as a cocaine addiction treatment, particularly as a therapeutic that helps prevent relapse of recovering cocaine addicts. Thus, the disclosure relates to a method reducing cocaine-primed reinstatement of drug-seeking behavior in a mammalian subject having had prior exposure to cocaine administering to the mammalian subject a cocaine hydrolase variant of BChE, preferably a plant-derived cocaine hydrolase variant of BChE. The disclosure also relates to treating acute cocaine toxicity by administering to a mammalian subject a cocaine hydrolase variant of BChE, preferably a plant-derived cocaine hydrolase variant of BChE.

In some aspects, the mammalian subject having had prior exposure to cocaine has had multiple self-induced exposures to cocaine. For example, the mammalian subject that self-administers cocaine on a regular basis. In certain implementations, the mammalian subject having had prior exposure to cocaine is an addict.

In some embodiments, the cocaine hydrolase variant of BChE is a mutated BChE comprising at least one substitution mutation at residue 119, residue 227, residue 287, residue 328, or residue 332, for example, at least two substitution mutations, at least three substitutions, or at least for substitution mutations. In certain embodiments, the combinations of mutations in a cocaine hydrolase variant of BChE are substitution mutations at residues 227, 287, 328, and 332; at residues 199, 287, 328, and 332; at residues 199, 227, 287, 328, and 332; or substitution mutations at residues 227, 287, 328, and 332, for example, the cocaine hydrolase variants specified in Table 3. In a particular embodiment, the cocaine hydrolase variant of BChE is PCocSH.

In certain aspects, the genome of the plant from which the cocaine hydrolase variant of BChE is produced is modified by cloning the synthetic gene encoding the cocaine hydrolase variant of human serum BChE into a TMV-based MagnICON vector system. In some aspects, the pBChE administered is a tetramer. In some aspects, the pBChE administered comprises a C-terminal 6x histidine tag. In certain implementations, the pBChE administered is conjugated to polyethylene glycol, for example, PEGylated (see Geyer et al., PNAS 2010). In other implementations, the pBChE is administered in a composition comprising other ingredients that are known to enhance the half-life and bioavailability of an enzyme.

In certain implementations, the pBChE is administered parenterally. For example, the pBChE may be administered to the mammalian subject intravenously, sublingually, subcutaneously, and/or transdermally. The pBChE may also be administered with a pulmonary route, for example, in an aerosolized form. The amount of pBChE administered to the mammalian subject is between 2 and 15 mg/kg body weight, for example, between 2 and 10 mg/kg body weight, between 3 and 10 mg/kg body weight, about 2 mg/kg body weight, at least 3 mg/kg body weight, about 3 mg/kg body weight, about 4 mg/kg body weight, about 5 mg/kg body weight, about 6 mg/kg body weight, about 7 mg/kg body weight, about 8 mg/kg body weight, about 9 mg/kg body weight, or about 10 mg/kg body weight.

In particular implementations of reducing cocaine-primed reinstatement of drug-seeking behavior in a mammalian subject having had prior exposure to cocaine, the pBChE is administered no more than a day before an incident of cocaine exposure, for example, no more than 20 hours, 12 hours, 10 hours, about 8 hours, about 6 hours, about 5 hours, about 4 hours, about 3 hours, about 2 hours, about 1 hour, about 30 minutes, about 15 minutes, about 10 minutes, or about 5 minutes before an incident of cocaine exposure. The mammalian subject's prior exposure to cocaine may be, in certain embodiment, at least one day from the administration of pBChE. In some aspects, the prior exposure to cocaine was at least one week, at least two weeks, about one week, or about two weeks from the administration of pBChE. Thus, in certain implementations, pBChE is administered to the mammalian subject when the cocaine-behavior of the subject has been extinguished for at least one day, at least one week, at least two weeks, about one day, about one week, or about two weeks.

In some implementations of reducing cocaine-primed reinstatement of drug-seeking behavior in a mammalian subject having had prior exposure to cocaine, the pBChE is administered to the mammalian subject once daily, weekly, or monthly. In some aspects, the pBChE is administered continuously or on the aforementioned schedule using a pump-liked device. When pBChE is administered continuous, the amount of the enzyme administered is between 2 and 15 mg/kg body weight/day, for example, between 2 and 10 mg/kg body weight/day, between 3 and 10 mg/kg body weight/day, about 2 mg/kg body weight/day, at least 3 mg/kg body weight/day, about 3 mg/kg body weight/day, about 4 mg/kg body weight/day, about 5 mg/kg body weight/day, about 6 mg/kg body weight/day, about 7 mg/kg body weight/day, about 8 mg/kg body weight/day, about 9 mg/kg body weight/day, or about 10 mg/kg body weight/day. In another implementation where pBChE is administered continuously, the amount of the enzyme administered is between 2 and 15 mg/kg body weight/week or between 2 and 15 mg/kg body weight/month, for example, between 2 and 10 mg/kg body weight/week, between 3 and 10 mg/kg body weight/week, about 2 mg/kg body weight/week, at least 3 mg/kg body weight/week, about 3 mg/kg body weight/week, about 4 mg/kg body weight/week, about 5 mg/kg body weight/week, about 6 mg/kg body weight/week, about 7 mg/kg body weight/week, about 8 mg/kg body weight/week, about 9 mg/kg body weight/day, about 10 mg/kg body weight/week, between 2 and 10 mg/kg body weight/month, between 3 and 10 mg/kg body weight/month, about 2 mg/kg body weight/month, at least 3 mg/kg body weight/month, about 3 mg/kg body weight/month, about 4 mg/kg body weight/month, about 5 mg/kg body weight/month, about 6 mg/kg body weight/month, about 7 mg/kg body weight/month, about 8 mg/kg body weight/month, about 9 mg/kg body weight/month, or about 10 mg/kg body weight/month.

In particular implementations of treating acute cocaine toxicity, pBChE is administered within 10 minutes after the mammalian subject is exposed to cocaine. In some embodiments, the pBChE is administered within a minute of the mammalian subject developing symptoms of acute cocaine toxicity. Symptoms of acute cocaine toxicity include cocaine-induced seizures or tremors, decreased motor activity, dragging feet, dystaxia, knuckle walking, labored breathing, and decreased locomotion. Symptoms of acute cocaine toxicity also include decreased responsiveness to stimuli and dyspnea.

Illustrative, Non-Limiting Example in Accordance with Certain Embodiments

The disclosure is further illustrated by the following examples that should not be construed as limiting. The contents of all references, patents, and published patent applications cited throughout this application, as well as the Figures, are incorporated herein by reference in their entirety for all purposes.

1. Plant production of a recombinant cocaine-hydrolyzing human BChE variant.

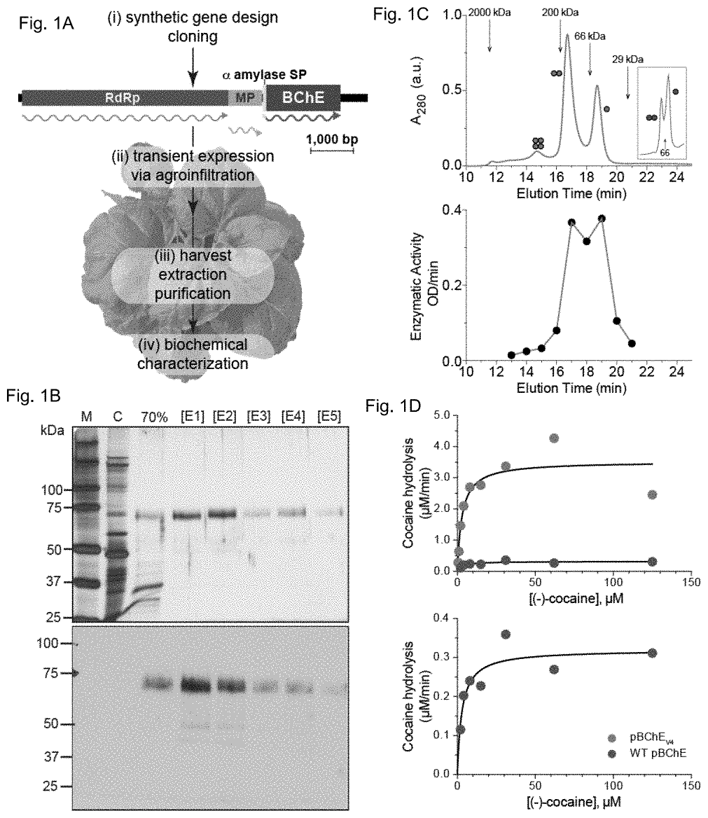

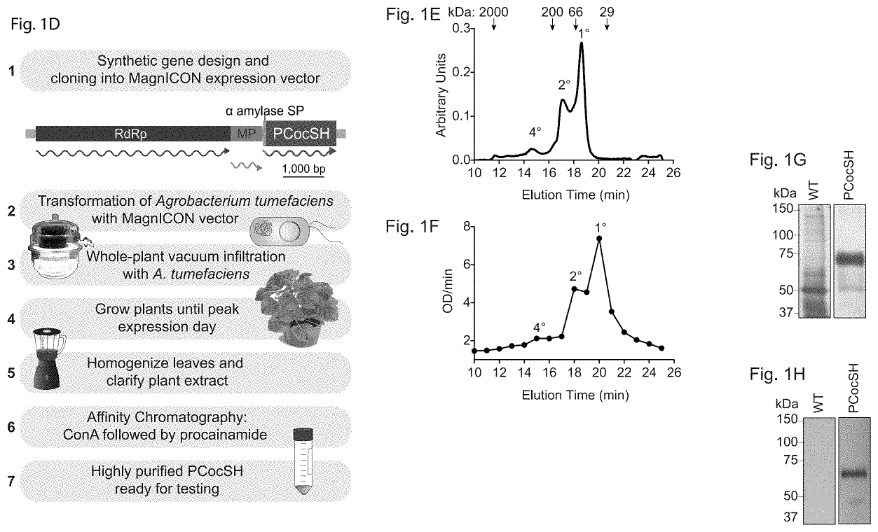

A low-cost, sustainable, source of recombinant BChE must be readily available to produce clinically useful quantities of BChE mutants. Rapid and high-level transient expression of foreign proteins in plants is needed to efficiently screen copious number of mutant variants, while maintaining the ability to ramp up production greatly when mutants of particular interest have been established. Mammalian expression systems have been used to produce cocaine hydrolase variants of BChE, but such platforms can be difficult and expensive to scale up. Plant-based recombinant protein production systems, in particular transient expression systems that make use of viral vectors (FIG. 1A), have advantages including reduced production costs, similar or cheaper downstream costs, as well as easy scalability.

Several research groups have been working on rational re-design of BChE into a cocaine hydrolase. The group led by Zhan used hybrid quantum mechanical/molecular mechanical (QM/MM) method-based predictions followed by validation through in vitro and in vivo experiments. This process provided evidence for a correlation between the measured catalytic efficiency of cocaine hydrolysis and the sum of the enzyme-substrate hydrogen-bonding distances within the first transition state. In successive papers Zhan et al. reported the further design of BChE variants with ever increasing catalytic efficiency.

Several of these variants (see Methods for a list of variants discussed here) have been produced using the deconstructed tobacco mosaic virus (TMV)-based expression system in plants (FIG. 1A). This virus-assisted transient expression system exploits plant viral vectors deconstructed for the rapid, industrial-scale expression of foreign proteins. In particular, pBChEᵥ₄ (A199S/F227A/S287G/A328W/Y332G) was reported to hydrolyze cocaine close to the upper limit set by substrate diffusion rates. Recently, another BChE variant with a 6th mutation, P285A, was reported with further two-fold better catalytic efficiency potentially bringing it to the diffusion-limited maximal theoretical ceiling.

The pBChEᵥ₄ was purified as previously reported for its WT counterpart. SDS-PAGE analysis of pBChEᵥ₄ revealed that it resolved with an apparent molecular mass of ˜65-70 kDa. This is similar to previously described plant-derived BChE variants and slightly smaller than the ˜85 kDa human BChE monomer, likely due to differences in glycosylation (FIG. 1B). When highly purified pBChEᵥ₄ was subjected to SEC-HPLC, about two thirds was dimeric (FIG. 1C). Most of the remainder were monomers, but low amounts of tetramers were also detected (FIG. 1C). Similar preparations of the WT enzyme, obtained through transient expression using the MagnICON system, showed inverse proportions of monomers and dimers (FIG. 1C inset). Interestingly, stable expression of WT enzyme in transgenic plants results in a substantial tetramer fraction.

The plant-derived pBChEᵥ₄ was examined closely for its ability to hydrolyze cocaine (FIG. 1D) and was found to have >2000-fold improved catalytic efficiency against that substrate (k_{cat}/K_{M}=1.9×10⁹ M⁻¹ min⁻¹) compared with the WT plant-derived enzyme (k_{cat}/K_{M}=9.0×10⁵ M⁻¹ min⁻¹). The higher efficiency is mostly due to a large increase in kcat of pBChEᵥ₄ as compared to WT pBChE (5805 min⁻¹ vs 2.6 min⁻¹, respectively) with nearly identical affinity to the substrate (K_{M}=3.0 μM vs K_{M}=2.9 μM, respectively). The catalytic efficiency of the plant-derived variant and its improvement over WT BChE are in agreement with reports of this same variant derived from other sources such as human embryonic kidney-293F cells.

In another experiment, PCocSH enzyme was transiently expressed in WT Nicotiana. benthamiana plants using the magnICON vector system based on deconstructed tobacco mosaic virus (FIG. 1E). All PCocSH prepared for animal studies was derived from plasmid pTM783 and has a C-terminal 6x histidine tag. The recombinant PCocSH was extracted as described previously. The preparation was subject to ConA purification and was eluted with five stepwise increasing concentrations of methyl-a-D-mannopyranoside (0.05 M, 0.1 M, 0.2 M, 0.5 M, and 1 M). Eluates showing similar degrees of purity were pooled, concentrated, and dialyzed against 20 mM sodium phosphate buffer, pH 7.5. The partially purified PCocSH was then subject to final polishing using batch procainamide (Sigma, catalogue no. P2240) affinity chromatography to remove any remaining contaminants. Proteins were released by stepwise elution as follows: 0.05 M NaCl, 0.5 M NaCl, 1 M NaCl, 1 M NaCl and 0.2 M Procainamide HCl, 1 M NaCl and 0.3 M Procainamide HCl. Eluates were dialyzed against PBS, pH 7.4 and eluates of similar purity were pooled and concentrated. After concentration, 0.02% sodium azide (NaN₃) was added to prevent unwanted organismal growth during storage, which was subsequently dialyzed out against PBS, pH 7.4 prior to use for animal experiments.

2. Cocaine hydrolase variants of BChE exhibit altered allosteric effects.

The specific residues changed to produce BChE-based cocaine hydrolases included those at the bottom of the catalytic gorge near the 7c-cation binding site (A328) and in the peripheral anionic site (Y332). Catalytic activity against (−)-cocaine was further improved through additional mutations to the oxyanion hole (A199), entrance to the gorge (S287) and non-active site residues participating in H-bonding (F227). Together, these changes result in increased catalytic efficiency against (−)-cocaine and potentially affect the enzyme's interactions with other substrates and ligands. Preliminary results with crude preparations revealed such effects. The following experiments were performed to determine whether there are other allosteric effects on the function of several plant-derived BChE variants.

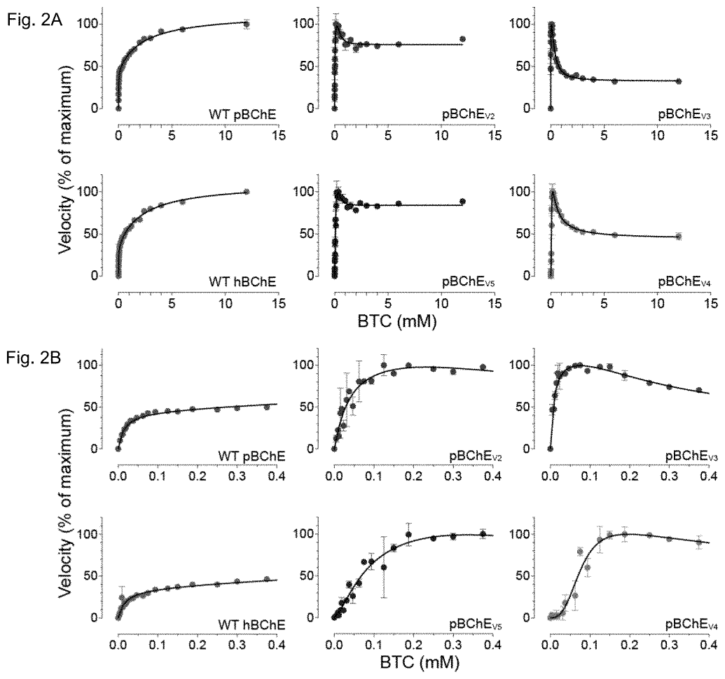

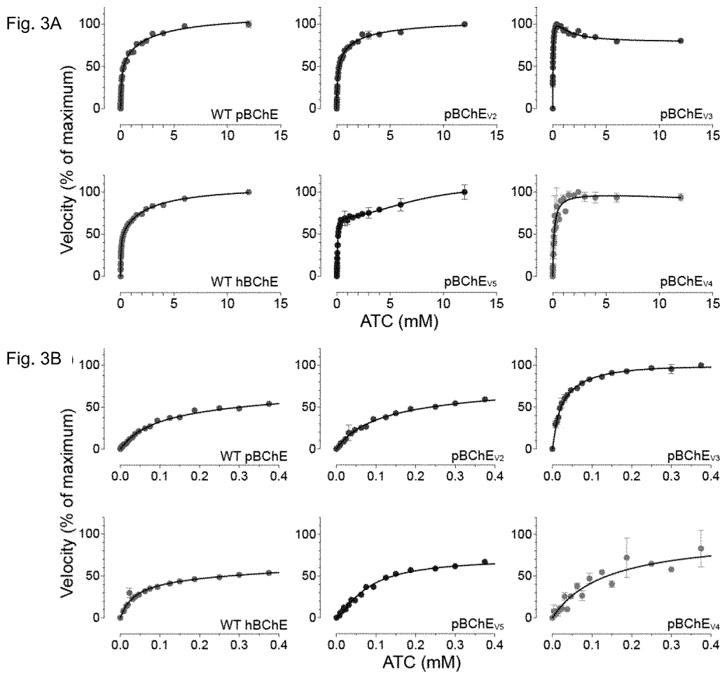

To rule out artifacts from the plant expression system, WT human plasma-derived (hBChE) was compared with WT plant-derived BChE (pBChE). The Michaelis-Menten constant (K_{M}) of WT hBChE and WT pBChE were determined with the substrate, butyrylthiocholine. Nonlinear regression analysis showed values of 16.8±2.9 μM and 14.6±1.4 μM respectively, similar to previous reports and essentially identical to each other (FIGS. 2A and 2B, FIGS. 3A and 3B, and Table 1). WT hBChE and WT pBChE also exhibited similar turnover numbers (k_{cat}=2.6×10⁴ min⁻¹ and k_{cat}=2.6×10⁴ min⁻¹, respectively) and catalytic efficiencies with BTC (k_{cat}/K_{M}=1.6×10⁹ M⁻¹ min⁻¹ and k_{cat}/K_{M}=1.8×10⁹ M⁻¹ min⁻¹, respectively; FIGS. 2A and 2B, Table 1).

Catalytic efficiency of pBChEᵥ₂, pBChEᵥ₃, and pBChEᵥ₄ toward BTC was reduced 100-, 37- and 5-fold, respectively, mostly due to a large reduction in the turnover number but also to small changes in the KM. An even larger drop (˜1000-fold) was observed in the catalytic efficiencies of pBChEᵥ₃ and pBChEᵥ₄ toward ATC, but not in the case of pBChEᵥ₂, which dropped only 3-fold. Of note, the catalytic efficiency of pBChEᵥ₅, toward both substrates remained equal to the WT enzyme (Table 1).

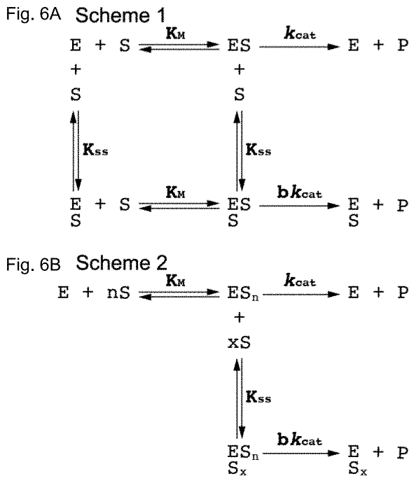

Human AChE and BChE exhibit characteristic allosteric effects due to low affinity substrate binding at the “peripheral site” (P-site) positioned near the entrance to the catalytic gorge. Despite the close homology between the two cholinesterases, their substrates exert opposite allosteric effects. AChE is inhibited by ACh concentrations above 5 mM, but BChE is stimulated by similar concentrations of ACh and BCh and their thioester analogues (ATC and BTC, FIGS. 2A, 2B, 3A, and 3B). This remains true regardless of the source of the enzyme, as both WT hBChE and WT pBChE exhibited typical substrate activation against BTC and ATC (FIGS. 2A and 2B, FIGS. 3A and 3B, and Table 1). A simple modification of the Michaelis-Menten model (Equation 1) results in an adequate steady-state description of the phenomena of substrate activation and inhibition in WT cholinesterases (Equation 2 (see Methods Section) and Scheme 1, FIG. 6A).

TABLE 1
Catalytic activity of WT BChE and cocaine hydrolase variants
against butyrylthiocholine and acetylthiocholine.

| | | WT | WT | | | | |
|---|---|---|---|---|---|---|---|
| Substrate | | hBChE | pBChE | pBChE_{V2} | pBChE_{V3} | pBChE_{V4} | pBChE_{V5} |
| BTC | Kinetic | Substrate | Substrate | Modified | Modified | Modified | Modified |
| | behavior | activation | activation | Hill | Hill | Hill | Hill |
| | k_{cat}/K_{M} | 1.6 × 10⁹ | 1.8 × 10⁹ | 1.8 × 10⁷ | 4.9 × 10⁷ | 3.4 × 10⁷ | 3.2 × 10⁸ |
| | (M⁻¹ | | | | | | |
| | min⁻¹) | | | | | | |
| | k_{cat} | 26241.2 | 25992.7 | 664.6 | 463.0 | 2806.0 | 27440.1 |
| | (min) | | | | | | |
| | K_{M} | 16.8 ± 2.9 | 14.6 ± 1.4 | 37.1 ± 21.8 | 9.5 ± 3.2 | 83.0 ± 21.5 | 86.2 ± 27.7 |
| | (μM) | | | | | | |
| | Kₛₛ | 2.2 ± 0.4 | 2.3 ± 0.3 | 0.4 ± 0.3 | 0.3 ± 0.1 | 0.4 ± 0.4 | 0.4 ± 0.2 |
| | (mM) | | | | | | |
| | b^{a} | 3.1 ± 0.2 | 2.5 ± 0.1 | 0.6 ± 0.2 | 0.3 ± 0.1 | 0.3 ± 0.2 | 0.7 ± 0.1 |
| | n^{b} | n.a. | n.a. | 1.1 ± 0.3 | 1.1 ± 0.2 | 2.8 ± 0.6 | 1.0 ± 0.2 |
| | x^{b} | n.a. | n.a. | 2.3 ± 2.0 | 2.3 ± 0.3 | 1.1 ± 0.6 | 1.5 ± 0.3 |
| | R² | 0.98 | 0.99 | 0.83 | 0.95 | 0.95 | 0.95 |
| ATC | Kinetic | Substrate | Substrate | Substrate | Modified | Michaelis- | Modified |
| | behavior | activation | activation | activation | Hill | Menten | Hill |
| | k_{cat}/K_{M} | 2.5 × 10⁸ | 9.6 × 10⁷ | 3.7 × 10⁷ | 9.1 × 10⁶ | 8.3 × 10⁶ | 2.1 × 10⁸ |
| | (M⁻¹ | | | | | | |
| | min⁻¹) | | | | | | |
| | k_{cat} | 9185.0 | 8490.1 | 3756.3 | 243.2 | 1093.3 | 16154.1 |
| | (min) | | | | | | |
| | K_{M} | 36.7 ± 3.8 | 89.2 ± 7.8 | 101 ± 13 | 26.8 ± 4.8 | 132 ± 13 | 77.0 ± 4.5 |
| | (μM) | | | | | | |
| | Kₛₛ | 2.3 ± 0.4 | 2.7 ± 0.5 | 2.4 ± 1.0 | 1.2 ± 0.4 | n.a. | 7.6 ± 3.3 |
| | (mM) | | | | | | |
| | b | 2.2 ± 0.1 | 1.9 ± 0.1 | 1.6 ± 0.1 | 0.7 ± 0.1 | n.a. | 1.6 ± 0.3 |
| | n | n.a. | n.a. | n.a. | 0.9 ± 0.1 | n.a. | 1.4 ± 0.1 |
| | x | n.a. | n.a. | n.a. | 1.4 ± 0.5 | n.a. | 2.3 ± 1.1 |
| | R² | 0.99 | 1.0 | 0.99 | 0.98 | 0.94 | 0.99 |

^{a}When b > 1, enzyme is exhibiting substrate activation; when b < 1, enzyme is exhibiting substrate inhibition; if b = 1, the enzyme is following Michaelis-Menten kinetics.
^{b}n and x represent the Hill coefficients. See Scheme 2 (FIG. 6B) and Equation 3 (see Methods section). Positive cooperativity is observed when either n > 1 or x > 1. Negative cooperativity is observed when n < or x < 1.

In striking contrast, hydrolysis of BTC by pBChEᵥ₃ and pBChEᵥ₄ revealed partial substrate inhibition (FIGS. 2A and 2B) reminiscent of the kinetics of human AChE with ACh as was previously reported for native and plant-derived human enzyme. But this inhibition (˜40%) was much weaker than that exhibited by AChE (>90%), and their respective peak activities were reached at BTC concentrations of approximately 70 μM and 230 μM respectively (FIGS. 2A and 2B).

Even more complex enzymatic behavior was exhibited by pBChEᵥ₂ and pBChEᵥ₅, which differ from each other only by, respectively, an alanine or a glycine residue at position 332 (FIGS. 2A and 2B). BTC at concentrations higher than approximately 125 to 375 μM had more limited inhibitory effect on these variants (about 20%, FIGS. 2A and 2B) as compared to pBChEᵥ₃ and pBChEᵥ₄. Interestingly, at still higher substrate concentrations (>2 mM) very slight but highly reproducible substrate activation re-appeared (FIGS. 2A and 2B).

Hydrolysis of the smaller substrate ATC also revealed differences between the four variants. In all tested variants, ATC has much weaker inhibitory effect on its hydrolysis. In fact, pBChEᵥ₂ and pBChEᵥ₅, which were somewhat inhibited by high concentrations of BTC, were clearly activated by high ATC concentrations, as was the case of WT hBChE or WT pBChE (FIGS. 3A and 3B). Still, pBChEᵥ₃ was inhibited at high concentrations of ATC (but not to the extent that BTC provoked), while pBChEᵥ₄ was not allosterically affected by the smaller substrate, exhibiting a hyperbolic Michaelis-Menten kinetic profile (FIGS. 3A and 3B).

The complex kinetic behavior of certain variants is reflected by the relatively poor fit between experimental data and the standard model for BChE and AChE's allosteric effects (Equation 2 (see Methods Sections) and Scheme 1, FIG. 6A). A closer look at hydrolysis rates at low BTC concentrations (FIG. 2B) showed a sigmoidal pattern as BTC concentrations rise. Sigmoidal behavior is characteristic for homo-oligomeric enzymes that exhibit cooperative binding of substrate molecules. Both BChE and AChE are oligomeric, tetramers and dimers being most common in vivo. But the common view is that oligomerization status does not affect the enzymatic properties of either enzyme. BChE purified from transgenic plants is about 50% tetrameric, while TMV-assisted transient-expression in plants yields a mixture of monomers and dimers with few tetramers (FIG. 1C). Thus, the mutations introduced into BChE to improve the enzyme's activity toward cocaine also affected the enzyme's subunit interactions, which in turn made the enzyme behave cooperatively. Nonetheless, even monomeric enzymes with multiple substrate binding-sites, like all cholinesterases, can also exhibit cooperative (or anticooperative) binding.

Including Hill coefficients describing cooperativity (or anticooperativity) into the standard analysis of uncompetitive inhibition provided an adequate model to describe the behavior of the BChE variants against BTC and ATC (Scheme 2, FIG. 6B). While the molecular mechanism is not yet established, the suggested model yields an estimate for factors that are assumed to be negligible for the WT enzymes (Scheme 2, FIG. 6B). Specifically, the model anticipates the possibility that binding of one substrate molecule at either the peripheral or the active site may alter the binding of a second molecule in the other site. The Hill coefficients (Table 1) demonstrate weak positive cooperativity, which would be particularly important at low substrate concentrations. At higher substrate concentrations, effects on k_{cat} are more prominent and result in the observed substrate inhibition (against BTC) and activation (against ATC). A non-equilibrium analysis of the interactions between the peripheral site and the active site, similar to the one offered by Rosenberry, should provide further insight into the mechanism involved here.

3. Dynamic Coupling Index (DCI) Analysis Predicts Allosteric Coupling Between the Pentavalent Mutations of pBChEᵥ₄ and its Active Site.

While all four variants exhibit novel enzymatic properties, the superior efficiency of cocaine hydrolysis of pBChEᵥ₄ compared to other variants (FIG. 1D, unpublished data and Zheng et al.), prompted further investigation of this variant. Specifically, the altered substrate preference, hydrolysis kinetics, and inhibitor sensitivity suggest that the mutated positions in pBChEᵥ₄ (A199S/F227A/S287G/A328W/Y332G), all but one of which are quite distal to the active site, may be allosterically linked to the catalytic locus. The “Dynamic Coupling Index” (DCI) analysis, which identifies residues exhibiting significant fluctuation upon perturbation of functionally important loci including the active catalytic site and other substrate binding sites in the protein, was used to test whether the mutation positions are allosterically linked to the catalytic locus.

DCI analysis identified positions that dynamically couple to residues of the catalytic triad, i.e. S198, E325 and H438. According to this analysis, positions exhibiting high DCI values present residues that are dynamically linked to the active site despite being far away from the catalytic residues.

In FIG. 4A, the % DCI values for human BChE upon perturbation of the three catalytic residues are color-coded within a spectrum of red-white-blue (from highest to lowest respectively). The five mutated positions of pBChEᵥ₄ variant (A199S, F227A, S287G, A328W and Y332G) are highly coupled to the catalytic triad. Conversely, a reciprocal analysis of perturbing the five mutated positions and measuring % DCI values for other residues show that the catalytic triad's residues are highly coupled to these five mutated positions (FIG. 4B). This reaffirms the hypothesis concerning dynamic interplay between these mutated positions and catalytic residues. Moreover, the strong dynamic coupling between mutational sites and the catalytic site suggests that mutations alter the conformational dynamics of the enzyme, leading to changes in enzymatic function.

The changes in catalytic properties of BChE variants can be partially attributed to the direct allosteric effect of peripheral amino-acid substitution on the catalytic triad suggested by DCI analysis (FIGS. 4A and 4B), These results also raise the possibility that such mutations affect the interactions between enzyme subunits—specifically they may lead to increased enzymatic cooperativity (FIGS. 2A, 2B, 3A, and 3B).

4. Dynamic flexibility index (DFI) analysis predicts global flexibility changes upon introduction of mutations.

To further substantiate the hypothesis that the mutation sites are allosterically linked to the catalytic locus and to provide mechanistic insights on how these five mutations lead to changes in enzymatic behavior, the conformational dynamics of the WT and the pBChEᵥ₄ variant was explored using a dynamic flexibility index (DFI). DFI computes the fluctuation response of a given position to the perturbations that occur at different parts of the protein using linear response theory, capturing the multi-dimensional effects when the protein structure is displaced out of equilibrium for example when interacting with small molecules or other cellular constituents. DFI allows us to identify and map flexible and rigid positions in the structure. DFI can be considered a measure of the local conformational entropy of a given position within the set of interactions governed by the 3D fold of the protein due to its ability to probe the conformational space of a protein at the residue level.

The DFI values of residues for WT hBChE and pBChEᵥ₄ was measured, and the % DFI profiles shows us the flexibility of the proteins in ranking order (FIG. 5A). Examining the flexibility of the monomer-monomer contact (binding) interfaces (FIG. 5B), it appears that the dimerization surface of pBChEᵥ₄ is less flexible in comparison with the WT counterpart. Rigidified monomer-monomer interface is often associated with increased affinity. The association constant for dimerization depends on the entropic cost at the binding interface: dimerization causes the binding interface to be more rigid and is therefore causing a decrease in entropy (negative entropy change associated with dimerization i.e. ΔS_{dimerization}<0). Because the entropy level associated with the WT contact surface is higher than in the mutant (i.e., the former is more flexible than the latter). Hence the entropic cost of dimerization is higher in WT than in the mutant (i.e. AS_{dimerization} of WT is more negative than that of the mutant). These results support the observation that preparations of pBChEᵥ₄ have higher proportion of dimers as compared to pBChE, which is mostly monomeric.

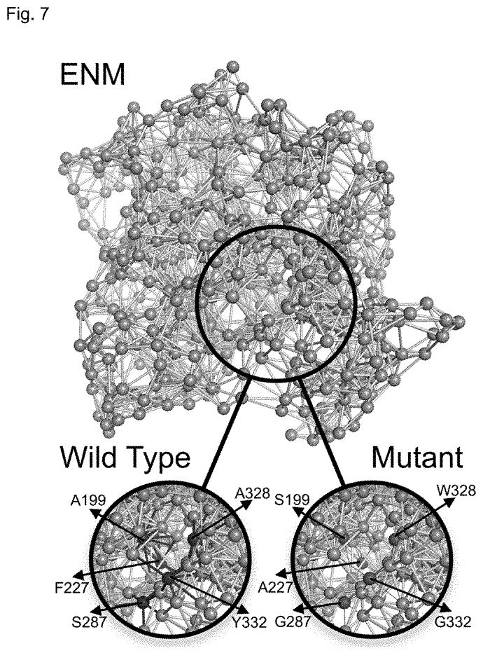

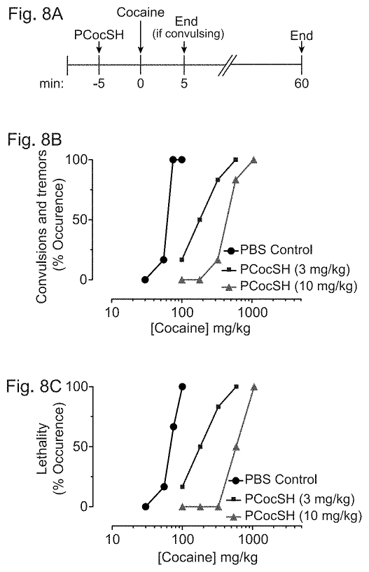

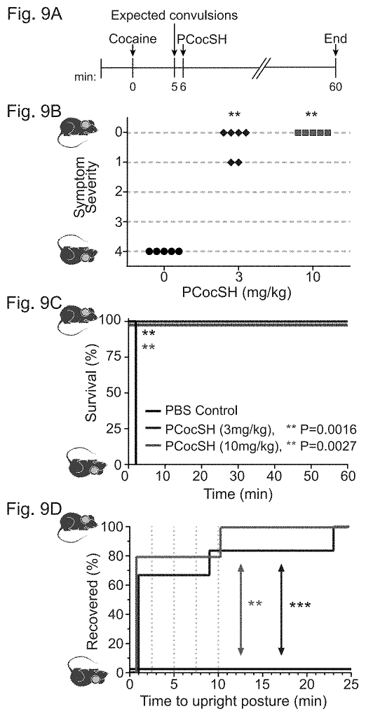

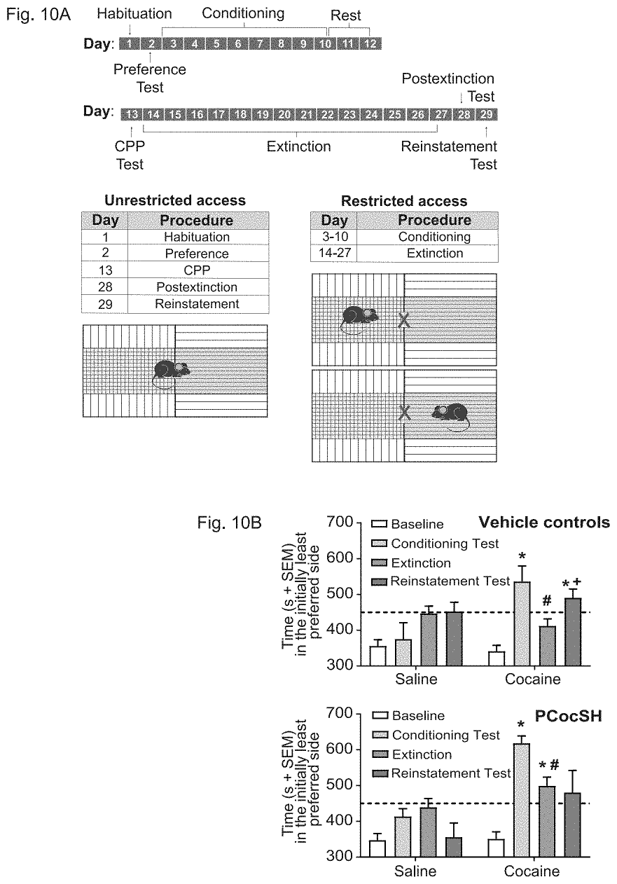

The oligomerization of WT BChE is usually not regarded as affecting the enzymatic properties of the enzyme. However, the sigmoidal nature of the enzyme kinetics observed here in the mutant variants (FIGS. 2A, 2B, 3A, and 3B) suggests a degree of cooperativity. If this is the case, dimerization should induce new conformational dynamics in the mutant but less so in the WT. To test this possibility, how dimerization may affect the dynamics of each monomeric subunit in WT and the mutant using DFI analysis was explored. The five residue substitutions are introduced into the Elastic Network Model (ENM) model (see Methods) at the core of the DFI analysis as changes in the spring constants of the harmonic oscillators interconnecting the alpha-carbons of the adjoining amino-acids (FIG. 7). In other words, since the mutations introduced local destabilization around the mutational sites, we modeled this effect as decreased spring constants for the interactions of the mutational positions (i.e. weakened harmonic interactions of the mutational sites).

With this approach, we can predict global changes in flexibility upon introduction of mutations. The local disruption due to the mutations not only introduce enhanced flexibilities at the mutational sites but can create a global flexibility change in all positions (i.e. change in DFI profile) due to network of interactions. In fact, it appears that the change in the flexibility of one region is compensated by the changes in flexibility of other regions. As could be expected based on the well-documented lack of cooperativity in BChE upon its oligomerization, the DFI profile of the WT hBCHE subunit in monomeric and dimeric form are quite similar (FIG. 5C). On the other hand, in the case of BChEV4 mutant, the DFI profile of subunit in the dimer form differs notably from that in monomeric form (FIG. 5D). This change suggests that dimerization induces new conformational dynamics. Interestingly, when we map the localized differences in the % DFI values between mutant and WT were mapped, it was observed that the mutations lead to enhanced flexibility near the gorge site in the dimer, but less so in the monomer (FIG. 5E). The peripheral anionic site (D70, N68, Q119, A227), cation-π domain (W82, A328), acyl pocket (L289 V288), and phenothiazine ring site (Y332, F329) exhibited increased flexibility upon mutations, while the rigid profile of the catalytic triad (S198, E235, H438) did not change.

The DFI analysis suggests that compared to the WT, BChE_{V4} should have a better propensity to dimerize and that within the mutant dimer there is an increase in flexibility near the gorge (FIG. 5E). We propose that changes in flexibility might facilitate propagation of conformational changes from one subunit to the other. Thus, at low substrate concentrations, binding of a substrate molecule on one of the subunits might positively affect substrate binding and/or turnover at the catalytic gorge of the other subunit, explaining the sigmoidal kinetic observed at low substrate concentrations (FIGS. 2B and 3B). At higher substrate concentrations, allosteric effects within each subunit may lead to inhibition countering the cooperative enhancement and explaining the observed partial substrate inhibition (FIGS. 2A and 3A).

### 5. Protection Study and Generation of Dose Response Curves.

The aim of this study was to develop a plant-based cocaine-hydrolyzing enzyme toward the generation of anti-cocaine therapeutics (FIGS. 1D-1H). In order to test whether PCocSH could counter cocaine-induced acute toxicity, the timeline of the intoxication and the cocaine dose-response in the presence or absence of the plant-derived enzyme first need to be determined. Highly purified PCocSH was prepared from plant material. Mice (n=6) were i.v. administered PCocSH (3 or 10 mg/kg) or vehicle control (PBS, pH 7.4) and challenged five minutes later with varying concentrations of cocaine (delivered i.p.).

The mammalian-derived enzyme was shown to be protective of a lethal 180 mg/kg dose of cocaine at a low enzyme dose of approximately 1 mg/kg in mice when delivered 1 minute before cocaine administration. In a separate study, a dose-response curve for seizures was generated using a higher enzyme dose of 10 mg/kg in rats when given 10 minutes prior to cocaine administration. The enzyme was administered five minutes prior to cocaine administration to determine if there was a dose-dependent protection afforded by the enzyme; therefore, both a low (3 mg/kg) and high (10 mg/kg) enzyme dosing regimen was used for this study (FIG. 8A).

Mice that were pre-treated with the low dose of PCocSH were almost fully protected from a cocaine dose (100 mg/kg) that killed 100% of mice in the vehicle control group (FIG. 8B). In fact, only one mouse of the 6 challenged with this cocaine dose showed signs of acute toxicity (FIG. 8B). This mouse seized for >60 seconds and was subsequently euthanized. None of the other mice (5/6) had any seizures, tremors, or other signs of acute toxicity. At 585 mg/kg cocaine, all mice seized for >60 seconds and were euthanized.

Mice were then treated with a higher enzyme dose of 10 mg/kg. Mice given this treatment did not exhibit any signs of cocaine-induced seizures or tremors at 100 or 180 mg/kg cocaine, which was 100% lethal in unprotected mice (FIG. 8B). At 180 mg/kg the mice initially had slightly decreased motor activity, which subsided within a few minutes. At 353 mg/kg most mice began to show neurologic symptoms (dragging feet, dystaxia, knuckle walking) but only 1 mouse exhibited seizures. At an even higher dose of 585 mg/kg, all mice (100%) exhibited neurologic symptoms described above as well as labored breathing and decreased locomotion. These mice had decreased responsiveness to stimuli, dyspnea, and decreased locomotion indicative of phase II acute cocaine toxicity rather than the premorbid phase III state. Therefore, even at doses nearly 20-fold higher than the typical rewarding cocaine dose of 30 mg/kg, animals are protected from advanced stages of acute cocaine toxicity. At this dose, 50% of the mice (3/6) recovered during the observation time and only three became moribund and had to be euthanized (FIG. 8C). With a protective PCocSH dose of 10 mg/kg, LD100 was reached only at 1055 mg/kg of cocaine, a >10-fold higher dose compared to unprotected control mice. This is equivalent to a mammalian (CHO cell) derived albumin-fused BChE variant's (A199S/S287G/A328W/Y332G) ability to protect rats and to the effect of the bacterial derived cocaine esterase (CocE) enzyme to protect mice and rats.

### 6. Rescue Study

To simulate a more clinically relevant scenario, this study determines whether PCocSH provided protection when delivered after cocaine-challenged subjects become symptomatic. To evaluate the potential for PCocSH to rescue mice from cocaine overdose, mice received the LD100 dose of cocaine as determined above (100 mg/kg, i.p.). Immediately upon onset of convulsions, mice were treated by i.v. injection through the tail vein with either low dose (3 mg/kg), high dose (10 mg/kg) of PCocSH or vehicle control (FIG. 9A).

As expected, all of the mice challenged by cocaine experienced severe effects of cocaine toxicity, except for one control mouse that displayed no severe symptoms and therefore was excluded from the study. The interval between cocaine administration and onset of convulsions was 4.8±0.5 minutes (n=17). One animal from the PCocSH (10 mg/kg) group died later while being restrained prior to receiving further treatment and was subsequently removed from the study.

The 100 mg/kg dose of cocaine was lethal to all 5 control (vehicle-treated) mice that exhibited cocaine-induced convulsions and were euthanized upon moribundity. Strikingly, all of the mice (n=6) that were treated with 3 mg/kg PCocSH soon after the onset of seizures survived (FIG. 9B). In all PCocSH-treated subjects, convulsions ceased within 1 minute of treatment (Table 2). Thus, PCocSH can reverse the acute effects of cocaine-induced toxicity in mice when given soon after the onset of convulsions. Moreover, most mice (4/6) fully recovered by the end of the observation period with only two of the mice (2/6) continuing to experience some mild symptoms (FIG. 9B). Delivery of the enzyme for these two mice took nearly 3 times longer than the other 4 mice due to the technically challenging nature of conducting an i.v. injection on a seizing animal. This prolonged time of seizing before receiving the full dose of the enzyme likely led to the lethargy observed in these mice.

TABLE 2
Effect of PCocSH treatment on mice following
cocaine-induced acute toxicity

| | Vehicle | PCocSH | PCocSH |
|---|---|---|---|
| | Control | 3 mg/kg | 10 mg/kg |
| # Mice | 5 | 6 | 5 |
| Time (min) from treatment | Never | <1 | <1 |
| until seizure subsided | | | |
| Time (min) from treatment | Never | 6.0 ± 3.6 | <1: 4/5 mice |
| until upright posture | | SEM | 10: 1/5 mice* |

*Mouse did not receive the full PCocSH dose during injection

Similar results of rescue were observed for mice (n=5) treated with the high dose of PCocSH (10 mg/kg) (FIG. 9C). All of these mice ceased convulsing in <1 min and all mice resumed upright posture in <10 min (FIG. 9D). Mice that received the full 10 mg/kg dose of PCocSH after onset of convulsions induced by 100 mg/kg cocaine exhibited an extremely rapid return to a normal state. In the time it took to replace the animal in its cage (<30 seconds), the mouse began to walk normally and resumed normal behavior.

In both the low and high enzyme groups, 67% of the mice regained upright posture in 1 minute or less following enzyme treatment, compared to 0% of the mice in the control group (FIG. 9D). All (100%) of the mice treated with complete doses of PCocSH (entire 3 or 10 mg/kg) which had exhibited cocaine-induced seizures were saved from subsequent death compared to 0% of mice which seized and received vehicle control.

### 7. Reinstatement of Cocaine-Conditioned Place Preference

Another highly problematic issue of cocaine use is addiction-related relapse after a period of abstinence. As the plant-derived enzyme was efficient enough to protect and rescue mice from lethal doses of cocaine, the next investigation evaluated the plant-derived enzyme's potential therapeutic use in managing cocaine addiction-related behavior using the mouse model of reinstatement of extinguished cocaine-CPP (FIG. 10A). Mice were tested for preference between two adjoining compartments of the CPP apparatus prior to conditioning (baseline), post-conditioning (CPP test), post-extinction, and after receiving a priming injection of cocaine or saline (reinstatement test).

On the reinstatement test day, mice were pretreated with an i.v. injection of vehicle or PCocSH. The omnibus ANOVA revealed a significant main effect of test day [F(3, 11)=16.8, P<0.001], and interactions between test day and conditioning drug [F(3, 11)=8.195, P<0.001] and test day and enzyme pretreatment given on the reinstatement test day [F(3, 11)=2.98, P <0.05] (FIG. 10B). To further analyze the interactions, two-way ANOVAs were conducted separately for the enzyme and vehicle (no enzyme) treatment groups. In the vehicle groups, there was a test day main effect [F(3,51)=7.04, P<0.001] and a drug by test day interaction [F(3, 51)=4.80, P<0.005]. The interaction was due to the saline control group showing no significant differences across the 4 tests [F(3,24)=1.75, n.s.], whereas the cocaine-conditioned group exhibited significant differences across tests [F(3, 27)=21.96, P <0.001]. The cocaine-conditioned group exhibited a significant increase in time spent in the drug-paired compartment during the CPP and reinstatement test days compared to baseline [t(11)=3.99, P<0.005 and t(9)=5.12, P<0.001], as well as a decrease in time spent in the drug-paired compartment during the extinction test compared to the CPP test. Furthermore, a planned comparison between the extinction and reinstatement tests indicated a significant difference in time spent in the drug-paired compartment between these two test days [t(9)=2.68, P<0.025].

The two-way ANOVA in the PCocSH-treated groups revealed significant main effects of test day [F(3,60)=12.58, P<0.001] and drug [F(1,20)=11.21, P<0.005], as well as a drug by test day interaction [F(3,60)=3.88, P<0.05]. Again, the interaction was due to the saline control group showing only a strong trend toward differences across the 4 tests [F(3,30)=2.91, P=0.051], whereas the cocaine-conditioned group exhibited significant differences across tests [F(3, 30)=12.06, P<0.001]. The cocaine-conditioned group exhibited a significant increase in time spent in the drug-paired compartment compared to baseline during the CPP test [t(11)=11.55, P<0.001] and the extinction test [t(11)=5.92, P<0.001] and a significant decrease in time spent in the drug-paired side during the extinction test compared to the CPP test [t(11)=5.10, P<0.001] (FIG. 10B). Importantly, the PCocSH-treated, cocaine-conditioned group failed to exhibit reinstatement of cocaine-CPP in response to the cocaine priming injection as there was no difference between time spent in the drug-paired side during the reinstatement test compared to the extinction test.

Previous reports on production of cocaine hydrolase variants of BChE relied on expression using mammalian cell cultures that are notoriously expensive to scale up. It is therefore important to note that the work presented here relied on our use of a cost-effective, sustainable and safe source of recombinant BChE (FIGS. 1D-1H). The system utilizes disarmed plant viral vectors to express and accumulate to relatively high levels (hundreds of milligrams to more than a gram of recombinant protein per kg of biomass) of PCocSH and similar variants of BChE. Among the advantages of such a transient expression system one can count reduced production costs, similar or cheaper drug-processing costs, as well flexible production scale to fit production needs. Moreover, plants constitute a malleable production system that can be engineered to have the protein product to present fully sialylated glycans that can support the necessary pharmacokinetic profile of the therapeutic.

### 8. Methods

a. DNA constructs

Previously, the inventors reported that the full-length human WT BChE gene (UniProt accession number P06276) was optimized for expression in N. benthamiana plants (pBChE). A synthetic gene encoding the A328W/Y332A mutant of pBChE (named here BChEᵥ₁) was used as the template for successive rounds of site-directed mutagenesis using the QuickChange method (Stratagene). The resulting mutants are listed in Table 3.

TABLE 3
Cocaine hydrolase variants of butyrylcholinesterase
used in this study.

| Name | Amino acid mutations | References |
|---|---|---|
| pBChE_{V2} | F227A/S287G/A328W/Y332A | Pancook, et al. 2003 |
| pBChE_{V3} | A199S/S287G/A328W/Y332G | Pan, et al. 2005 |
| pBChE_{V4} | A199S/F227A/S287G/A328W/Y332G | Xue, et al. 2013 |
| pBChE_{V5} | F227A/S287G/A328W/Y332G | Brimijoin and |
| | | co-workers, |
| | | unpublished |

The mutated genes were verified by DNA sequencing. A C-terminal hexahistidine tag was added to the plant-expression optimized variants of BChE. The resulting constructs were then cloned into a deconstructed tobacco mosaic virus (TMV)-based plant expression vector (MagnICON, kind gift of Nomad Inc.) to be used in Agrobacterium tumefaciens-mediated transient expression in N. benthamiana.

b. Transient Recombinant Protein Production in Plants and Purification

An outline of the expression strategy is shown in FIGS. 1A and 1E. All expression vectors were electroporated into A. tumefaciens strain GV3101 electro-competent cells. Transformed strains were screened via antibiotic selection as well as colony screen PCR and only positive colonies were used for downstream studies. Bacteria cultures were grown at 30° C. until mid-logarithmic phase, pelleted by centrifugation at 4,500×g for 20 min at room temperature and then resuspended in infiltration buffer (10 mM 2-(N-morpholino)ethanesulfonic acid (MES), 10 mM magnesium sulfate heptahydrate, pH 5.5). Plants were infected either by needle-less syringe injection or by whole-plant vacuum infiltration. Leaves infiltrated with each variant were harvested at the respective day of peak expression as determined in previous reports.

All extraction and purification procedures were carried out at 4° C. Large-scale protein preparations were extracted from plant leaf tissue by blending in the presence of 50 mM sodium phosphate, 150 mM sodium metabisulfite, 1 mM EDTA, pH 8.0. Extract was filtered through double-layer miracloth and centrifuged at 22,000×g for 30 min followed by pH adjustment to pH 5.0 and further clarification by ammonium sulfate precipitation. The pellet was resuspended in cold 1X phosphate buffered saline (PBS) and dialyzed overnight against lx PBS, pH 7.4 to remove salts and sodium metabisulfite. The clarified protein preparation was then subjected to sequential affinity chromatography steps with Concanavalin-A-Sepharose followed by procainamide affinity chromatography as previously described.

For PCocSH, the gene was codon-optimized for plant expression and relevant side-directed mutations as made. The gene was then cloned into a transient plant expression vector system such as magnICON vector system based on deconstructed tobacco mosaic virus. This system was transformed into Agrobacteria tumefaciens. The PCocSH enzyme was transiently expressed in WT Nicotiana benthamiana plants. Specifically, to infiltrate the bacteria into the intercellular spaces of the leaf tissue, entire plants were submerged in A. tumefaciens solution, and a vacuum was applied. (FIGS. 1A and 1D). All PCocSH prepared for animal studies was derived from plasmid pTM783 and has a C-terminal 6x histidine tag.

The recombinant PCocSH was extracted as described previously. Briefly, following infiltration, plants were returned to the growth chamber or greenhouse until the day post-inoculation that results in peak recombinant protein expression (established during small-scale time course experiments). On the peak expression day, leaf tissue was homogenized, and total soluble proteins was filtered and clarified. PCocSH was purified by sequential Concanovalin A and procainamide purification steps. Following dialysis into a suitable buffer, the purified PCocSH product can be stored for several years at 4° C. and used for testing.

Specifically, the preparation was subject to ConA purification and was eluted with five stepwise increasing concentrations of methyl-a-D-mannopyranoside (0.05 M, 0.1 M, 0.2 M, 0.5 M, and 1 M). Eluates showing similar degrees of purity were pooled, concentrated, and dialyzed against 20 mM sodium phosphate buffer, pH 7.5. The partially-purified PCocSH was then subject to final polishing using batch procainamide (Sigma, catalogue no. P2240) affinity chromatography to remove any remaining contaminants. Proteins were released by stepwise elution as follows: 0.05 M NaCl, 0.5 M NaCl, 1 M NaCl, 1 M NaCl and 0.2 M Procainamide HCl, 1 M NaCl and 0.3 M Procainamide HCl. Eluates were dialyzed against PBS, pH 7.4 and eluates of similar purity were pooled and concentrated. After concentration, 0.02% sodium azide (NaN3) was added to prevent unwanted organismal growth during storage which was subsequently dialyzed out against PBS, pH 7.4 prior to use for animal experiments.

c. Enzymatic Assays

To evaluate cocaine hydrolysis, a sensitive radiometric assay was used. Briefly, [3H](−)-cocaine labeled on the benzene ring (50 Ci/mmol), purchased from PerkinElmer Life Sciences (Boston, Mass.), was used as a substrate with varying concentrations of (−)-cocaine. In the presence of enzyme this reaction proceeded at room temperature (25° C.) until stopped by the addition of 0.02 M HCl. Any neutralized, liberated, labeled benzoic acid was then extracted with a toluene-based fluor and measured by scintillation counting. On the other hand, the substrate would fractionate into the aqueous phase and would not generate scintillation. Enzyme concentrations in the reaction mix were 800 ng/100 μL (1.21×10⁻¹ μM) for WT pBChE and 4 ng/100 μL (6.06×10⁻⁴ μM) for pBChEᵥ₄.

Choline ester hydrolysis activity was evaluated by a modified Ellman assay^{8,63,64}. Activity was measured using either butyrylthiocholine iodide (BTC, Sigma) or acetylthiocholine iodide (ATC, Sigma) at 30° C. in a Spectramax 190 spectrophotometer (Molecular Devices). Total soluble protein levels were determined by the Bradford protein assay (Bio-Rad Protein Assay Reagent, Bio-Rad). The assay was conducted in 96-well plate format over varying concentrations of BTC or ATC in final well volume of 200 μL. To account for product formed by substrate self-hydrolysis, initial velocity of non-enzymatic hydrolysis was subtracted from initial velocity of the matched enzyme-catalyzed reactions and reaction rates were then plotted as a function of substrate concentration.

Data were plotted using GraphPad Prism software, which was also used to fit the data by non-linear regression. The following models were fitted.

For Michaelis-Menten kinetics we used Equation (1)

For substrate inhibition/activation, we used Equation (2), following the model in Scheme 1 (FIG. 7), as was suggested by Radić and coworkers.

The Radić model ascribes the allosteric effect of substrate binding at the peripheral binding site that causes a change in the catalytic rate bk_{cat}. When b>1 we encounter substrate activation (WT BChE, see below), when b<1 we encounter substrate inhibition and when b=1 we have a Michaelian enzyme (Equation 1). K6ₛₛ is the dissociation constant of the peripheral site.

Velocity vs substrate concentration data of some of the BChE variants described here fitted well to a model initially suggested by LiCata and Allewell⁶⁶ for aspartate transcarbamylase (Scheme 2, FIG. 6B). This model describes the reaction in terms of uncompetitive substrate inhibition/activation and cooperative substrate binding with characteristic Hill coefficients. The equation describing this model is Equation (3).

The Hill coefficients n and x need not be integers. Values greater than one describe cooperativity, while values of less than one describe anti-cooperativity. The parameters b and Kₛₛ function in the same way as in Equation (2).

$$\begin{matrix}v = ( \frac{{V}_{max} [ S ]}{{K}_{M} + [ S ]} ) & ( Equation 1 ) \\ v = ( \frac{1 + b [ S ] / {K}_{ss}}{1 + [ S ] / {K}_{ss}} ) ( \frac{{V}_{max}}{1 + {K}_{M} / [ S ]} ) & ( Equation 2 ) \\ v = \frac{{V}_{max} ( 1 + {b [ S ]}^{x} / {K}_{ss}^{x} )}{1 + ( {K}_{M}^{n} / {[ S ]}^{n} ) + ( {[ S ]}^{x} / {K}_{ss}^{x} )} & ( Equation 3 )\end{matrix}$$

d. Inhibition

Inhibition studies were conducted with the OPs paraoxon (diethyl (4-nitrophenyl) phosphate) and iso-OMPA (N [bis(propan 2 ylamino)phosphoryloxy-(propan 2 ylamino) phosphoryl]propan 2-amine), the carbamate neostigmine bromide ([3 (dimethylcarbamoyloxy) phenyl]-trimethylazanium;bromide), or the reversible bisquaternary inhibitor BW284c51 (BW, [4 [5 [4-[dimethyl(prop-2-enyl)azaniumyl]phenyl]-3-oxopentyl]phenyl]-dimethyl-prop-2-enylazanium; dibromide). The four inhibitors were purchased from Sigma (St Louis, Mo.).

Preparations of BChE and variants thereof were incubated in 96-well plate format with indicated concentrations of the inhibitors for 30 min at room temperature followed by activity measurements based on modified Ellman assay using 1 mM BTC as the substrate. IC50 values were determined by non-linear regression (GraphPad Prism) fit according to Equation (4).

The inhibition rate constant (ki) of pBChEV4 treated with paraoxon was determined as previously described.

Inhibition curves were statistically analyzed by the extra sum-of-squares F test (GraphPad Prism) together and were found to be significantly different from each other. Following up with individual comparisons to WT pBChE revealed statistical significance in all except the following: paraoxon inhibition of WT pBChE vs WT hBChE, Iso-OMPA inhibition of WT pBChE vs pBChEV4, and neostigmine inhibition of WT pBChE vs pBChEV3.

$$residual BChE activity = \frac{100}{1 + {10}^{( log [ I ] - {logIC}_{50} )}} \qquad ( Equation 4 )$$

e. Dynamic Flexibility Index (DFI) analysis

Dynamic flexibility index (DFI) metric is based on the Perturbation Response Scanning method (PRS) that couples covariance matrix of residue displacement with linear response theory (LRT).

PRS was originally based on the Elastic Network Model (ENM). In ENM, protein is viewed as an elastic network, in which each amino acid is represented by C-alpha position, and a harmonic interaction is assigned to pairs of amino-acids within a specified cutoff distance. Simply put, in the WT BChE, two residues that are interacting with each other are represented by a harmonic interaction with the same spring constant (lower left in FIG. 8). In contrast, a mutation at a given position is considered to destabilize the interactions of the mutational site. Thus, this destabilization is introduced in the ENM as a decrease in spring constant providing a loss in interaction strength with mutated positions (lower right in FIG. 8), the mutation positions S199, A227, G287, W328, and G332 are shown as spheres in the right inset).

A random Brownian kick was applied in PRS as a perturbation to a single residue in the chain one at a time, sequentially. This perturbation mimics the external forces exerted on the protein through interactions with another protein, another biological macromolecule or small molecule ligand, in silico. The perturbation cascades through the residue interaction network and may introduce conformational changes in the protein. Then, we compute the response fluctuation profile of all other residues to the perturbation as linear response using Equation (5) where F is a unit random force on selected residues, H-1 is the inverse of the Hessian matrix and AR is the positional displacements of the N residues of the protein in three dimensions.

The response fluctuation profile is used to calculate the DFI scores using Equation (6), where [ΔR\hat{ }j]_i is the response fluctuation amplitude of position i, upon perturbing position j. Thus, the DFI of position i is the total fluctuation response of position i upon perturbing all positions in the chain one at a time. The DFI value for each position is normalized to the overall intrinsic flexibility of the protein chain. High and low DFI scores could be interpreted as dynamically flexible sites and rigid (hinge) sites, respectively. The DFI scores can be converted into percentile ranking scores, namely % DFI.

$$\begin{matrix}{[ Δ R ]}_{3 Nx 1} = {{[ H ]}_{3 Nx 3 N}^{- 1} [ F ]}_{3 Nx 1} & ( Equation 5 ) \\ {DFI}_{i} = \frac{{\sum }_{j = 1}^{N} {⌈ Δ {R}^{j} ⌉}_{i}}{{\sum }_{i = 1}^{N} {\sum }_{j = 1}^{N} {⌈ Δ {R}^{j} ⌉}_{i}} & ( Equation 6 )\end{matrix}$$

f. Dynamic Coupling Index (DCI) Analysis

The position-specific metric DCI uses PRS methodology to identify the residues that are allosterically linked to functionally critical positions through residue fluctuation dynamics for a given protein. The index DCI computes whether this position exhibits a higher fluctuation response to a perturbation that occurred at functionally critical sites (e.g. binding sites or catalytic site) compared to the perturbations at the other sites of the chain. It is measured as the ratio of average fluctuation responses of a given residue j upon perturbations of functionally critical sites to the average response of residue j upon perturbations placed on all other residues using

$${DCI}_{i} = \frac{{\sum }_{j = {N}_{functional}}^{{N}_{functional}} {\vert Δ {R}^{j} \vert}_{i} / {N}_{functional}}{{\sum }_{i = 1}^{N} {\sum }_{j = 1}^{N} {⌈ Δ {R}^{j} ⌉}_{i} / N} \qquad ( Equation 7 )$$

In Equation (7), |ΔR^{j}|ᵢ is the response fluctuation profile of residue j upon perturbation of residue i. The numerator is the average mean square fluctuation response obtained over the perturbation of the functionally critical residues N_{functional}; the denominator is the average mean square fluctuation response over all residues.

These DCI profiles can also be converted into rank profiles, which are labeled as % DCI profiles. The positions that have higher % DCI values are functionally important residues that are not linked to the functional residues by direct covalent bonds or non-covalent interactions (e.g. hydrogen bonding and van der Waals interactions), but are allosterically communicating over longer distances (i.e. allosteric dynamic coupling) via residues that form extensive interaction networks.

g. SDS-PAGE and Western Blot

Plant-derived protein preparations were resolved by SDS-PAGE on 8% polyacrylamide gels followed by staining with Pierce Silver Stain Kit (Thermoscientific). In parallel, protein was transferred to nitrocellulose membrane and decorated with rabbit polyclonal anti-hBChE antibodies (kindly provided by Dr. Oksana Lockridge) and anti-rabbit IgG-Horse Radish Peroxidase secondary antibodies (Santa Cruz Biotechnology) followed by chemiluminescence analysis using western blotting luminol reagent (Santa Cruz Biotechnology).

h. Size Exclusion HPLC

SEC-HPLC fractionation of purified preparations of pBChEV4 was carried out as previously described using Alliance HPLC (Waters) with a Shodex KW-803 column (8×300 mm, Kawasaki) 8. All samples were run in filtered, degassed mobile phase buffer (20 mM Na₂HPO₄/NaH₂PO₄, pH 8.0, 200 mM NaCl, 0.04% NaN₃) at a flow rate of 0.5 mL/min. Molecular mass standards used were blue dextran (2000 kDa) and the proteins β-amylase (200 kDa), bovine serum albumin (66 kDa) and carbonic anhydrase (29 kDa). Fractions were collected and analyzed for cholinesterase activity by the modified Ellman assay.

i. Animals and Intravenous Enzyme and Intraperitoneal Drug Administration

Black C57BL/6 male mice weighing 25-30 g at the start of the experiment were housed 5 mice per cage with free access to food and water in a temperature-controlled colony room with a reversed 12-h light:dark cycle and allowed at least one week to acclimate upon arrival to the housing facility.

All animal experiments were performed in accordance to protocols approved by the Institutional Care and Use Committee of Arizona State University. Moribund animals, and all animals by the end of the observation period were euthanized by CO2 asphyxiation followed by a secondary method of euthanasia (cervical dislocation). Moribundity was defined as prolonged seizures (≥120 s), or more than two shorter seizures within 2 minutes.

For intravenous (i.v.) injections of highly purified PCocSH or vehicle control (PBS, pH 7.4), mice were placed in a commercial mouse restrainer. The exposed tail was cleaned with an alcohol wipe and a U-100 insulin syringe (28G1/2) needle (Becton Dickinson) was used for injection into one of the tail veins. The i.v. injection volume did not exceed 5 μL/g body weight. The (−)-cocaine hydrolchloride (RTI International Triangle Park, N.C., USA) was dissolved in sterile 0.9% saline and filtered through a 0.2 μm membrane. Mice were released from the restraint as soon as the injection was complete and sterile gauze and pressure were applied to the injection site to stop any bleeding. For intraperitoneally (i.p.) injections of cocaine or vehicle (0.9% saline), the same kind of syringe as described above was used. Injection volume did not exceed 10 μL/g mouse.

j. Protection Experiment

Vehicle control (1× PBS, pH 7.4) or purified PCocSH (3 or 10 mg/kg) was administered i.v. (tail) 5 minutes before i.p. administration of varying doses of (−)-cocaine. The presence or absence of convulsions, lethality, and general observations were recorded for 60 minutes after cocaine administration or, if applicable, until euthanasia criteria were met. First, the dose response to cocaine in absence of any enzyme protection was tested using the following cocaine doses: 0, 30, 55, 75 and 100 mg/kg. Higher varying doses of cocaine (100, 180, 325, 585, and 1055 mg/kg) were needed to observe toxicity when PCocSH (3 or 10 mg/kg) was administered 5 minutes prior. For each experimental group n=6 animals at the beginning of the experiment.

k. Rescue from Acute Cocaine Toxicity

Vehicle control (1× PBS, pH 7.4) or purified PCocSH (3 or 10 mg/kg) was administered i.v. (tail) 5 minutes before i.p. administration of varying doses of (−)-cocaine. The presence or absence of convulsions, lethality, and general observations were recorded for 60 minutes after cocaine administration or, if applicable, until euthanasia criteria were met. First, the dose response to cocaine in absence of any enzyme protection was tested using the following cocaine doses: 0, 30, 55, 75 and 100 mg/kg. Higher varying doses of cocaine (100, 180, 325, 585, and 1055 mg/kg) were needed to observe toxicity when PCocSH (3 or 10 mg/kg) was administered 5 minutes prior. For each experimental group n=6 animals at the beginning of the experiment.

l. Conditioned Place Preference

Conditioned place preference (CPP) experiments were conducted using Plexiglas two-compartment chambers. Each chamber contained a removable partition that separated the chamber into two equal-sized compartments, each measuring 35 cm×24 cm×31 cm high. One compartment had a wire 1×1 cm grid floor and alternating black and white vertical stripes (2 cm wide) on the walls. The other had a parallel bar floor (5 mm diameter) and alternating black and white horizontal stripes (2 cm wide) on the walls. Both compartments had corncob bedding beneath the floors.

The testing room was dimly lit with two overhead lamps, each containing a 25 W light bulb. A camera (Panasonic WV-CP284, color CCTV, Suzhou, China) was mounted 101 cm above the center of the apparatus to record the sessions. A WinTV 350 personal video recorder (Hauppage, N.J., USA) captured the live video and encoded it to MPEG streams. A modified version of TopScan Software (Clever Sys., Inc. Reston, Va., USA) tracked the animal's body parts (e.g., nose, head, center of body, forepaws, base of tail, etc.) to estimate the position of the whole body when some parts were not in view in order to yield measures of time spent in each compartment.

Habituation day (Day 1): Mice were transported to the conditioning room and allowed free excess to both sides of the apparatus for 10 minutes to habituate them to the novel environment.

Pre-conditioning/baseline test (Day 2): Initial baseline side preference was assessed by allowing mice to freely explore both sides of the chamber without a middle partition for 15 minutes with locomotor activity and time spent in each compartment being recorded. Time spent on each side of the chamber was used to determine side preference for each mouse. During subsequent conditioning, a biased design was used such that the drug-paired compartment was each animal's least preferred side. Using baseline data, mice were divided into two drug groups that received either saline (controls) or cocaine prior to being confined to their initially nonpreferred side, with specific side preference equally distributed among the groups.

Conditioning phase (Days 3-10): Sessions were held at the same time each day for eight consecutive days. On odd numbered conditioning days, half of the mice in each group received saline and were placed into the non-drug-paired compartment for 30 min and on even numbered conditioning days these mice received their assigned drug [either saline or cocaine (10 mg/kg, i.p.)] and were placed into the drug-paired compartment for 30 minutes. The other half of the mice underwent the same procedure in the opposite order, such that they received their assigned drug prior to placement into the drug-paired compartment on odd numbered days and saline prior to placement into the non-drug-paired compartment on even numbered days.

CPP test (Day 13): Three days after the last conditioning session, mice were tested for the expression of CPP as previously done for the pre-conditioning test. The 72-hour interval between the last conditioning day and the test eliminated residual cocaine and recovery influences and allowed the consolidation of long-term memory of drug reward.

Extinction (Days 14-27): In order to extinguish cocaine-CPP, mice received repeated exposures to the drug-paired compartment without cocaine while also receiving exposure to the non-drug-paired compartment on alternate days to avoid relative novelty on the test day. Extinction sessions occurred at the same time of day across fourteen consecutive days. Mice received a saline injection prior to each session and were subsequently placed into the appropriate compartment for 20 minutes.

Post-extinction test (Day 28): To determine extinction of cocaine-CPP, mice were tested identically to the previous pre-conditioning and CPP tests.

Cocaine-induced reinstatement test (Day 29): On the day following the post-extinction test, mice were assessed for cocaine-induced reinstatement of CPP with and without treatment with PCocSH. To this end, mice were placed into a restrainer and injected i.v. through the tail vein with saline or enzyme at a 3 mg/kg dose at a volume of 5 μg body weight. Five minutes later, a priming injection of cocaine (10 mg/kg) or saline was administered (i.p.) at a volume of 10 μg body weight. The number of mice per group were as follows: vehicle control (no PCocSH) with vehicle control (no cocaine) (n=9), vehicle control (no PCocSH) with cocaine (n=10), PCocSH with vehicle control (no cocaine) (n=12), and PCocSH with cocaine (n=12). After the priming injection, the animals were immediately placed into the apparatus for 15 minutes with unrestricted access to both compartments. Locomotor activity and time spent in the cocaine- and saline-paired compartments were recorded.

m. Statistical Analysis

For protection and rescue experiments, statistical analyses were carried out using the GraphPad Prism software. Log-rank (Mantel-Cox) test was used to determine significance of the difference between treatment and control group survival and recovery curves. Data from the behavioral toxicity studies (percentage of mice showing lethality) were analyzed with two-tailed Fisher's exact probability test. The criterion for significance was set at p<0.05.

For CPP experiments, a mixed factor ANOVA with drug and enzyme treatments as between subjects factors and test day (baseline, CPP, extinction, reinstatement) as a repeated measure was performed. Interactions were further analyzed using smaller ANOVAs and paired-sample t-tests with Bonferroni correction. In addition, planned paired-samples t-tests between the extinction and reinstatement test days were performed to test the hypothesis that PCocSH would prevent cocaine-primed reinstatement.

### REFERENCES CITED

Alvarez-Garcia, D. & Barril, X. Relationship between Protein Flexibility and Binding: Lessons for Structure-Based Drug Design. J Chem Theory Comput 10, 2608-2614, doi:10.1021/ct500182z (2014).
Anker J J, et al. (2012) Cocaine Hydrolase Encoded in Viral Vector Blocks the Reinstatement of Cocaine Seeking in Rats for 6 Months. Biological psychiatry 71(8):700-705.
Atilgan, A. R. et al. Anisotropy of fluctuation dynamics of proteins with an elastic network model. Biophys J 80, 505-515, doi:10.1016/50006-3495(01)76033-X (2001).
Atilgan, C. & Atilgan, A. R. Perturbation-response scanning reveals ligand entry-exit mechanisms of ferric binding protein. PLoS Comput Biol 5, e1000544, doi:10.1371/journal.pcbi.1000544 (2009).
Atilgan, C., Gerek, Z. N., Ozkan, S. B. & Atilgan, A. R. Manipulation of conformational change in proteins by single-residue perturbations. Biophys J 99, 933-943, doi:10.1016/j.bpj.2010.05.020 (2010).
Barak, D. et al. Allosteric modulation of acetylcholinesterase activity by peripheral ligands involves a conformational transition of the anionic subsite. Biochemistry 34, 15444-15452 (1995).
Blong, R. M., Bedows, E. & Lockridge, O. Tetramerization domain of human butyrylcholinesterase is at the C-terminus. The Biochemical journal 327 (Pt 3), 747-757 (1997).
Boeck, A. T., Schopfer, L. M. & Lockridge, O. DNA sequence of butyrylcholinesterase from the rat: expression of the protein and characterization of the properties of rat butyrylcholinesterase. Biochem Pharmacol 63, 2101-2110 (2002).
Brimijoin S, et al. (2008) A Cocaine Hydrolase Engineered from Human Butyrylcholinesterase Selectively Blocks Cocaine Toxicity and Reinstatement of Drug Seeking in Rats. Neuropsychopharmacology: official publication of the American College of Neuropsychopharmacology 33(11):2715-2725.
Brimijoin, S., Shen, M. L. & Sun, H. Radiometric solvent-partitioning assay for screening cocaine hydrolases and measuring cocaine levels in milligram tissue samples. Anal Biochem 309, 200-205 (2002).
Butler, B. M., Gerek, Z. N., Kumar, S. & Ozkan, S. B. Conformational dynamics of nonsynonymous variants at protein interfaces reveals disease association. Proteins 83, 428-435, doi:10.1002/prot.24748 (2015).
Carmona, G. N. et al. Butyrylcholinesterase accelerates cocaine metabolism: in vitro and in vivo effects in nonhuman primates and humans. Drug Metab Dispos 28, 367-371. (2000).
Carroll M, Gao Y, Brimijoin S, & Anker J (2011) Effects of cocaine hydrolase on cocaine self-administration under a PR schedule and during extended access (escalation) in rats. Psychopharmacology 213(4):817-829.
Chen V P, et al. (2015) Plasma butyrylcholinesterase regulates ghrelin to control aggression. Proc Natl Acad Sci USA 112(7):2251-2256.
Chen X, et al. (2016) Long-acting cocaine hydrolase for addiction therapy. Proc Natl Acad Sci USA 113(2):422-427.
Chen, X. et al. Kinetic characterization of a cocaine hydrolase engineered from mouse butyrylcholinesterase. Biochem J 466, 243-251, doi:10.1042/BJ20141266 (2015).
Chen, X., Fang, L., Liu, J. & Zhan, C. G. Reaction pathway and free energy profiles for butyrylcholinesterase-catalyzed hydrolysis of acetylthiocholine. Biochemistry 51, 1297-1305, doi:10.1021/bi201786s (2012).
Cohen-Barak 0, et al. (2015) Safety, pharmacokinetics, and pharmacodynamics of TV-1380, a novel mutated butyrylcholinesterase treatment for cocaine addiction, after single and multiple intramuscular injections in healthy subjects. J Clin Pharmacol 55(5):573-583.
Connors N J & Hoffman R S (2013) Experimental treatments for cocaine toxicity: a difficult transition to the bedside. J Pharmacol Exp Ther 347(2):251-257.
Cooper Z D, et al. (2006) Rapid and Robust Protection against Cocaine-Induced Lethality in Rats by the Bacterial Cocaine Esterase. Molecular pharmacology 70(6):1885-1891.
Decker, M. Novel inhibitors of acetyl- and butyrylcholinesterase derived from the alkaloids dehydroevodiamine and rutaecarpine. Eur J Med Chem 40, 305-313 (2005).
Doctor, B. P. & Saxena, A. Bioscavengers for the protection of humans against organophosphate toxicity. Chem Biol Interact 157-158, 167-171 (2005).
Evron, T. et al. Plant-derived human acetylcholinesterase-R provides protection from lethal organophosphate poisoning and its chronic aftermath. Faseb J 21, 2961-2969 (2007).
Felder, C. E., Harel, M., Silman, I. & Sussman, J. L. Structure of a complex of the potent and specific inhibitor BW284C51 with Torpedo californica acetylcholinesterase. Acta Crystallogr D Biol Crystallogr 58, 1765-1771 (2002).
Gao Y, et al. (2008) An albumin-butyrylcholinesterase for cocaine toxicity and addiction: Catalytic and pharmacokinetic properties. Chemico-biological interactions 175(1-3):83-87.
Gao, D. & Zhan, C. G. Modeling evolution of hydrogen bonding and stabilization of transition states in the process of cocaine hydrolysis catalyzed by human butyrylcholinesterase. Proteins 62, 99-110, doi:10.1002/prot.20713 (2006).
Gao, D. et al. Computational design of a human butyrylcholinesterase mutant for accelerating cocaine hydrolysis based on the transition-state simulation. Angew Chem Int Ed Engl 45, 653-657, doi:10.1002/anie.200503025 (2006).
Gerek, Z. N. & Ozkan, S. B. Change in allosteric network affects binding affinities of PDZ domains: analysis through perturbation response scanning. PLoS Comput Biol 7, e1002154, doi:10.1371/journal.pcbi.1002154 (2011).
Geyer B C, Woods R R, & Mor T S (2008) Increased organophosphate scavenging in a butyrylcholinesterase mutant. Chem Biol Interact 175(1-3):376-379.
Geyer, B. C. et al. Plant-derived human butyrylcholinesterase, but not an organophosphorous-compound hydrolyzing variant thereof, protects rodents against nerve agents. Proc Natl Acad Sci USA 107, 20251-20256, doi:1009021107 [pii] 10.1073/pnas.1009021107 (2010).
Geyer, B. C. et al. Purification of Transgenic Plant-Derived Recombinant Human Acetylcholinesterase-R. Chem Biol Interact 157-158, 331-334 (2005).
Geyer, B. C. et al. Transgenic plants as a source for the bioscavenging enzyme, human butyrylcholinesterase Plant Biotechnol J 8, 873-886, doi:PBI515 [pii] 10.1111/j.1467-7652.2010.00515.x (2010).
Geyer, B. C. et al. Translational control of recombinant human acetylcholinesterase accumulation in plants. BMC Biotechnol 7, 27 (2007).
Geyer, B. C., Woods, R. R. & Mor, T. S. Increased organophosphate scavenging in a butyrylcholinesterase mutant. Chem Biol Interact 175, 376-379 (2008).
Gilgun-Sherki Y, et al. (2016) Placebo-controlled evaluation of a bioengineered, cocaine-metabolizing fusion protein, TV-1380 (AlbuBChE), in the treatment of cocaine dependence. Drug and alcohol dependence 166:13-20.
Inaba T, Stewart DJ, & Kalow W (1978) Metabolism of cocaine in man. Clinical pharmacology and therapeutics 23(5):547-552.
Inaba, T., Stewart, D. J. & Kalow, W. Metabolism of cocaine in man. Clin Pharmacol Ther 23, 547-552 (1978).
Khan, S. B. et al. Butyrylcholinesterase inhibitory guaianolides from Amberboa ramosa. Arch Pharm Res 28, 172-176 (2005).
Kim, H. et al. A hinge migration mechanism unlocks the evolution of green-to-red photoconversion in GFP-like proteins. Structure 23, 34-43, doi:10.1016/j.str.2014.11.011 (2015).
Ko MC, et al. (2007) Cocaine esterase: interactions with cocaine and immune responses in mice. The Journal of pharmacology and experimental therapeutics 320(2):926-933.
Kumar, A., Butler, B. M., Kumar, S. & Ozkan, S. B. Integration of structural dynamics and molecular evolution via protein interaction networks: a new era in genomic medicine. Curr Opin Struct Biol 35, 135-142, doi:10.1016/j.sbi.2015.11.002 (2015).
Kumar, A., Glembo, T. J. & Ozkan, S. B. The Role of Conformational Dynamics and Allostery in the Disease Development of Human Ferritin. Biophys J 109, 1273-1281, doi:10.1016/j.bpj.2015.06.060 (2015).
Larrimore K E, et al. (2013) Plants as a source of butyrylcholinesterase variants designed for enhanced cocaine hydrolase activity. Chem Biol Interact 203(1):217-220.
Larrimore K E, et al. (2017) Plant-expressed cocaine hydrolase variants of butyrylcholinesterase exhibit altered allosteric effects of cholinesterase activity and increased inhibitor sensitivity. Sci Rep 7(1):10419.
Li, Z. et al. A Rigid Hinge Region Is Necessary for High-Affinity Binding of Dimannose to

Cyanovirin and Associated Constructs. Biochemistry 54, 6951-6960, doi:10.1021/acs.biochem.5b00635 (2015).
LiCata, V. J. & Allewell, N. M. Is substrate inhibition a consequence of allostery in aspartate transcarbamylase? Biophys Chem 64, 225-234 (1997).
Lockridge, O. Review of human butyrylcholinesterase structure, function, genetic variants, history of use in the clinic, and potential therapeutic uses. Pharmacol Ther 148, 34-46, doi:10.1016/j.pharmthera.2014.11.011 (2015).
Loizzo, M. R., Tundis, R. & Menichini, F. Natural products and their derivatives as cholinesterase inhibitors in the treatment of neurodegenerative disorders: an update. Curr Med Chem 15, 1209-1228 (2008).
Masson, P., Xie, W., Froment, M. T. & Lockridge, O. Effects of mutations of active site residues and amino acids interacting with the Omega loop on substrate activation of butyrylcholinesterase. Biochim Biophys Acta 1544, 166-176 (2001).
Mionetto, N., Morel, N., Massoulie, J. & Schmid, R. D. Biochemical determination of insecticides via cholinesterases .1. Acetylcholinesterase from rat brain: Functional expression using a baculovirus system, and biochemical characterization. Biotechnology Techniques 11, 805-812, doi:Doi 10.1023/A:1018425224892 (1997).
Mor T S (2015) Molecular pharming's foot in the FDA's door: Protalix's trailblazing story. Biotechnol Lett.
Mor, T. S., Sternfeld, M., Soreq, H., Arntzen, C. J. & Mason, H. S. Expression of recombinant human acetylcholinesterase in transgenic tomato plants. Biotechnol. Bioeng. 75, 259-266 (2001).
Nevin Gerek, Z., Kumar, S. & Banu Ozkan, S. Structural dynamics flexibility informs function and evolution at a proteome scale. Evol Appl 6, 423-433, doi:10.1111/eva.12052 (2013).
Pan Y, et al. (2005) Computational redesign of human butyrylcholinesterase for anticocaine medication. Proc Natl Acad Sci USA 102(46):16656-16661.
Pancook, J. D. et al. Application of directed evolution technology to optimize the cocaine hydrolase activity of human butyrylcholinesterase. Faseb Journal 17, A565-A565 (2003).
Radic, Z., Pickering, N. A., Vellom, D. C., Camp, S. & Taylor, P. Three distinct domains in the cholinesterase molecule confer selectivity for acetyl- and butyrylcholinesterase inhibitors. Biochemistry 32, 12074-12084 (1993).
Rosenberry, T. L. Strategies to resolve the catalytic mechanism of acetylcholinesterase. J Mol Neurosci 40, 32-39, doi:10.1007/s12031-009-9250-3 (2010).
Saxena, A. et al. Prophylaxis with human serum butyrylcholinesterase protects Gottingen minipigs exposed to a lethal high-dose of sarin vapor. Chem Biol Interact 238, 161-169, doi:10.1016/j.cbi.2015.07.001 (2015).
Saxena, A., Hur, R. S., Luo, C. & Doctor, B. P. Natural monomeric form of fetal bovine serum acetylcholinesterase lacks the C-terminal tetramerization domain. Biochemistry 42, 15292-15299, doi:10.1021/bi030150x (2003).
Schneider J D, et al. (2014) Expression of human butyrylcholinesterase with an engineered glycosylation profile resembling the plasma-derived orthologue. Biotechnol J 9(4):501-510.
Schneider J D, et al. (2014) Oligomerization status influences subcellular deposition and glycosylation of recombinant butyrylcholinesterase in Nicotiana benthamiana. Plant Biotechnol J 12(7):832-839.
Shafferman, A. et al. Substrate inhibition of acetylcholinesterase: residues affecting signal transduction from the surface to the catalytic center. EMBO J. 11, 3561-3568 (1992).
Shram M J, et al. (2015) Assessment of Pharmacokinetic and Pharmacodynamic Interactions

Between Albumin-Fused Mutated Butyrylcholinesterase and Intravenously Administered Cocaine in Recreational Cocaine Users. J Clin Psychopharmacol 35(4):396-405.
Stewart D J, Inaba T, Lucassen M, & Kalow W (1979) Cocaine metabolism: cocaine and norcocaine hydrolysis by liver and serum esterases. Clin Pharmacol Ther 25(4):464-468.
Stewart D J, Inaba T, Tang B K, & Kalow W (1977) Hydrolysis of cocaine in human plasma by cholinesterase. Life Sciences 20(9):1557-1563.
Sun H, Pang Y-P, Lockridge O, & Brimijoin S (2002) Re-engineering Butyrylcholinesterase as a Cocaine Hydrolase. Molecular pharmacology 62(2):220-224.
Sun H, Shen M L, Pang Y-P, Lockridge O, & Brimijoin S (2002) Cocaine Metabolism Accelerated by a Re-Engineered Human Butyrylcholinesterase. Journal of Pharmacology and Experimental Therapeutics 302(2):710-716.
Sun, H. et al. Predicted Michaelis-Menten complexes of cocaine-butyrylcholinesterase. Engineering effective butyrylcholinesterase mutants for cocaine detoxication. J Biol Chem 276, 9330-9336, doi:10.1074/jbc.M006676200 (2001).
Topp E, et al. (2016) The case for plant-made veterinary immunotherapeutics. Biotechnol Adv.
Velan, B. et al. The effect of elimination of intersubunit disulfide bonds on the activity, assembly, and secretion of recombinant human acetylcholinesterase. Expression of acetylcholinesterase Cys-580—Ala mutant. J Biol Chem 266, 23977-23984 (1991).
Wilkins M R, et al. (1999) Protein identification and analysis tools in the ExPASy server. Methods Mol Biol 112:531-552.
Xie, W. et al. An improved cocaine hydrolase: the A328Y mutant of human butyrylcholinesterase is 4-fold more efficient. Mol Pharmacol 55, 83-91 (1999).
Xue L, et al. (2011) Design, Preparation, and Characterization of High-Activity Mutants of Human Butyrylcholinesterase Specific for Detoxification of Cocaine. Molecular Pharmacology 79(2):290-297.
Xue L, et al. (2013) Catalytic activities of a cocaine hydrolase engineered from human butyrylcholinesterase against (+)- and (−)-cocaine. Chemico-biological interactions 203(1):57-62.
Xue L, et al. (2013) Preparation and in vivo characterization of a cocaine hydrolase engineered from human butyrylcholinesterase for metabolizing cocaine. Biochem J 453(3):447-454.
Yang W, Xue L, Fang L, Chen X, & Zhan C-G (2010) Characterization of a high-activity mutant of human butyrylcholinesterase against (−)-cocaine. Chemico-Biological Interactions 187(1-3):148-152.
Zhan, M., Hou, S., Zhan, C. G. & Zheng, F. Kinetic characterization of high-activity mutants of human butyrylcholinesterase for the cocaine metabolite norcocaine. Biochem J 457, 197-206, doi:10.1042/BJ20131100 (2014).
Zhang T, et al. (2017) Clinical Potential of an Enzyme-based Novel Therapy for Cocaine Overdose. Scientific reports 7(1):15303.
Zheng F & Zhan CG (2011) Enzyme-therapy approaches for the treatment of drug overdose and addiction. Future 3(1):9-13.
Zheng F, et al. (2008) Most Efficient Cocaine Hydrolase Designed by Virtual Screening of Transition States. Journal of the American Chemical Society 130(36):12148-12155.
Zheng F, et al. (2014) A highly efficient cocaine-detoxifying enzyme obtained by computational design. Nat Commun 5:3457.
Zheng, F. et al. Design of high-activity mutants of human butyrylcholinesterase against (−)-cocaine: structural and energetic factors affecting the catalytic efficiency. Biochemistry 49, 9113-9119, doi:10.1021/bi1011628 (2010).
Zlebnik, N. E. et al. Long-term reduction of cocaine self-administration in rats treated with adenoviral vector-delivered cocaine hydrolase: evidence for enzymatic activity. Neuropsychopharmacology 39, 1538-1546, doi:10.1038/npp.2014.3 (2014).
Zou, T., Risso, V. A., Gavira, J. A., Sanchez-Ruiz, J. M. & Ozkan, S. B. Evolution of conformational dynamics determines the conversion of a promiscuous generalist into a specialist enzyme. Mol Biol Evol 32, 132-143, doi:10.1093/molbev/msu281 (2015).

We claim:

1. A method of reducing cocaine-primed reinstatement of drug-seeking behavior in a mammalian subject having had prior exposure to cocaine, the method comprising: administering to the mammalian subject a plant-expressed butyrylcholinesterase (BChE, UniProt accession number P06276) comprising a substitution mutation at residue 199, residue 227, residue 287, residue 328, and residue 332.

2. The method of claim 1, wherein the mammalian subject has had multiple self-induced exposures to cocaine.

3. The method of claim 2, wherein the substitution mutations are A199S, F227A, S287G, A328W, and Y332G.

4. The method of claim 2, wherein the plant-expressed BChE is a tetramer.

5. The method of claim 2, wherein the plant-expressed BChE comprises a C-terminal 6x histidine tag.

6. The method of claim 2, wherein the plant-expressed BChE is administered to the mammalian subject parenterally.

7. The method of claim 5, wherein the plant-expressed BChE is administered intravenously.

8. The method of claim 5, wherein the plant-expressed BChE is administered sublingually.

9. The method of claim 5, wherein the plant-expressed BChE is administered subcutaneously.

10. The method of claim 5, wherein the plant-expressed BChE is administered transdermally.

11. The method of claim 2, wherein cocaine-seeking behavior of the mammalian subject has been extinguished for at least one day.

12. The method of claim 2, wherein cocaine-seeking behavior of the mammalian subject has been extinguished for at least one week.

13. The method of claim 2, wherein cocaine-seeking behavior of the mammalian subject has been extinguished for at least two weeks.

14. The method of claim 2, wherein the mammalian subject is administered at least 3 mg plant-expressed BChE per kg body weight.

15. The method of claim 2, wherein the mammalian subject is administered 3-10 mg plant-expressed BChE per kg body weight.

16. The method of claim 1, wherein the mammalian subject is administered 3 mg plant-expressed BChE per kg body weight.

17. The method of claim 1, wherein the mammalian subject is an addict.

18. A method of treating acute cocaine toxicity, the method comprising:
administering to the mammalian subject a plant-expressed butyrylcholinesterase (BChE, UniProt accession number P06276) comprising a substitution mutation at residue 199, residue 227, residue 287, residue 328, and residue 332.

19. The method of claim 18, wherein the plant-expressed BChE is administered within 10 minutes after the mammalian subject is exposed to cocaine.

20. The method of claims 18, wherein the plant-expressed BChE is administered within a minute of the mammalian subject developing symptoms of acute cocaine toxicity.
